(19) United States
(12) Patent Application Publication
(10) Pub. No.: US 2018/0356318 A1
(43) Pub. Date: Dec. 13, 2018

(54) METHODS FOR CONTROLLING CHEMICAL REACTION KINETICS AND INTERACTION TIME IN LARGE SYSTEMS

(71) Applicant: Massachusetts Institute of Technology, Cambridge, MA (US)

(72) Inventors: Kwanghun Chung, Cambridge, MA (US); Evan Murray, Cambridge, MA (US); Jae Hun Cho, Cambridge, MA (US)

(73) Assignee: Massachusetts Institute of Technology, Cambridge, MA (US)

(21) Appl. No.: 15/780,875

(22) PCT Filed: Dec. 2, 2016
(86) PCT No.: PCT/US2016/064538

§ 371 Date: Jun. 1, 2018

Related U.S. Application Data
(60) Provisional application No. 62/262,366, filed on Dec. 2, 2015.

(51) Int. Cl. G01N 1/30; G01N 33/53; A01N 1/02

(52) U.S. Cl. CPC G01N 1/30; G01N 2021/1765; A01N 1/0231; G01N 33/5306

(57) ABSTRACT

This disclosure provides methods for preserving samples such as tissue samples and rendering such preserved samples mechanically and chemically stable throughout repeated rounds of labeling and imaging for a plurality of targets.

### RELATED APPLICATION

This application claims the benefit under 35 U.S.C. § 119(e) of U.S. Provisional Application Ser. No. 62/262,366 entitled “METHODS FOR CONTROLLING CHEMICAL REACTION KINETICS AND INTERACTION TIME IN LARGE SYSTEMS” filed on Dec. 2, 2015, the entire contents of which are incorporated by reference herein.

### BACKGROUND OF INVENTION

Biological systems are comprised of vast numbers of molecules, cell types, and intricate tissue organizations (Alivisatos et al.. 2013; Kasthuri et al., 2015; Yuste, 2015). Understanding the complex interactions of these components is essential for many fields of biology and often requires high-dimensional information across many scales. Although it is desirable to obtain such information from the same tissue due to large individual variations, combined measurement of many molecular and anatomical traits have not been heretofore achieved despite the remarkable success of current pioneering methods, such as array tomography (Micheva et al., 2010; Rah et al., 2013).

Rapidly evolving tissue-clearing techniques may enable multiplexed labeling and imaging of intact samples using light microscopy (Chung et al., 2013; Chung and Deisseroth, 2013; Renier et al., 2014; Richardson & Lichtman, 2015; Susaki et al., 2014). For instance, the CLARITY technique has demonstrated three rounds of immunostaining of mouse brain tissue (Chung et al., 2013). However, the polyacrylamide-based framework loses structural integrity upon repeated exposure to the elution condition. Recent reports also suggest that preservation of antigenicity in the CLARITY method may not be optimal (Renier et al., 2014). Furthermore, the necessary tissue-gel hybridization step requires delivery of charged thermal initiators with limited diffusivity and stability. This necessity imposes a limit on the tissue size that can be processed without the use of transcardial perfusion.

### SUMMARY OF INVENTION

Provided herein, inter alia, is a scalable and generalizable tissue-processing method for imaging, including proteomic imaging, of intact biological systems such as but not limited to biological samples such as but not limited to tissue sections, in vitro tissue cultures, in vitro 3-dimensional organ culture, etc. The method involves a process referred to herein as “SWITCH” (system-wide control of interaction time and kinetics of chemicals). SWITCH tightly controls a broad range of chemical reactions in tissue processing via a set of buffers including a SWITCH-On buffer and a SWITCH-Off buffer. The SWITCH-On buffer facilitates chemical reactions between exogenous chemicals and endogenous biomolecules, such as proteins, and the SWITCH-Off buffer suppresses these reactions. SWITCH-mediated fixation renders the tissue of interest sufficiently heat- and chemical-resistant while preserving tissue architecture, as well as preserving native molecules and their antigenicity to a degree suitable for subsequent multiplexed imaging such as proteomic imaging. The preserved sample, such as a preserved tissue, can be rapidly cleared at high temperature without damage. The method does not require perfusion, and it is thus applicable to both animal and large human samples. In molecular labeling of the processed samples, SWITCH controls probe-target binding kinetics to improve probe penetration depth and the uniformity of molecular labeling. The method does not require any special equipment or reagents.

As described in greater detail in the Examples, a banked post-mortem human tissue sample that is treated with the SWITCH process was able to undergo a minimum of 22 rounds of molecular labeling, and precise co-registration of multiple datasets at single cell resolution was achieved. The method also facilitated extraction of a wide range of system variables, such as various cell types and microvasculature from a single sample. These tissue processing methods, optionally together with a volumetric co-registration algorithm, can be readily adopted by most laboratories for scalable imaging such as proteomic imaging of intact biological systems.

The methods provided herein facilitate imaging including proteomic imaging by providing (1) high preservation of endogenous biomolecules and their antigenicity, (2) high structural integrity, and (3) minimal tissue damage during repeated cycles of destaining, labeling, and imaging processes. As demonstrated in the Examples, only 3-5% protein loss was observed for tissue samples preserved according to these methods after clearing. This is significantly better than protein loss in control tissues (30-40%) and polyacrylamide tissue gels (10-20%).

Furthermore when the preserved samples were labeled with antibodies for specific targets, surprisingly 86 out of 90 antibodies tested were compatible and thus able to bind to the sample yielding meaningful results about the presence and level of the target.

Thus, in one aspect, the disclosure provides a method for preserving a biological sample comprising contacting a biological sample with a crosslinker under a first condition that reduces crosslinking activity at least 100-fold, and exposing the sample to a second condition that restores crosslinking activity, wherein the first condition minimally comprises a low pH, and the second condition minimally comprises a neutral pH. In some embodiments, the first condition is a condition that reduces crosslinking activity at least 100-fold or 150-fold or 200-fold relative to a physiological condition minimally defined as being at about neutral pH. In some embodiments, the first condition is a condition that reduces crosslinking activity at least 100-fold or 150-fold or 200-fold relative to the crosslinking activity that occurs in the second condition.

In another aspect, the disclosure provides a method for processing a sample comprising exposing a sample to a first condition comprising a crosslinker and low pH, and exposing the sample to a second condition comprising neutral pH, and then inactivating the crosslinker.

In another aspect, the disclosure provides a method for preserving a sample comprising exposing a sample to a crosslinker under a first condition that reduces crosslinking activity at least 100-fold, and exposing the sample to a second condition that restores crosslinking activity, and then inactivating the crosslinker. In some embodiments, the first condition comprises low pH. In some embodiments, the second condition comprises neutral pH.

In some embodiments, low pH is a pH of about 3. In some embodiments, neutral pH is a pH of about 7.

In another aspect, the disclosure provides a method for preserving a biological sample comprising contacting a biological sample with a crosslinker under a first condition that reduces crosslinking activity at least 100-fold, and exposing the sample to a second condition that restores crosslinking activity, wherein the first condition has a pH that is lower than the pH of the second condition. The first condition pH may be 1, 2, 3, 4 or more pH units lower than the pH of the second condition. In some embodiments, the first condition pH is a neutral pH and the second condition pH is a higher or basic pH. In some embodiments, the first condition pH is in the range of about 6.5 to about 8, or about 7 to about 8, and the second condition pH is higher than the first condition pH. The second condition pH may be about 8 to about 11 provided it is higher than the first condition pH. The second condition pH may he about 8.5 to about 11, or about 9 to about 11, or about 9.5 to about 11, or about 10 to about 11, or about 10.5 to about 11, provided it is higher than the first condition pH.

In some embodiments, the first condition comprises a temperature of about 4-10° C. In some embodiments, the first condition comprises a temperature of about 4° C. In some embodiments, the second condition comprises a temperature of about 4-10° C. In some embodiments, the second condition comprises a temperature of about 4° C. In some embodiments, the second condition comprises a temperature of about 25-40° C. In some embodiments, the second condition comprises a temperature of about 37° C.

In some embodiments, the sample is exposed to the crosslinker under the first condition for 1-2 days. In some embodiments, the sample is exposed to the crosslinker under the second condition for 1-4 days. 1-2 days, or 2-3 days. In some embodiments, the sample is exposed to the second condition for 1-10 hours.

In some embodiments, the crosslinker is a bifunctional crosslinker. In some embodiments, the crosslinker is a multifunctional crosslinker. In some embodiments, the crosslinker is ethylene glycol diglycidyl ether (EGDGE), dipropylene glycol diglycidyl ether (GE23), 1,4-butanediol diglycidyl ether (GE21), glycerol polyglycidyl ether (EX-313), polyglycerol-3-polyglycidyl ether (GE38), or glutaraldehyde (GA).

In some embodiments, the sample is contacted with a solution of about 4-10% crosslinker under the first condition. In some embodiments, the sample is contacted with a solution of about 1-4% crosslinker under the second condition.

In some embodiments, the crosslinker is inactivated by addition of glycine and acetamide.

In some embodiments, the sample is a human tissue sample. In some embodiments, the sample is an animal tissue sample. In some embodiments, the sample is a brain, liver, lung, kidney or spinal cord sample.

In another aspect, the disclosure provides a method for imaging a sample comprising
(1) preserving a sample according to any of the foregoing methods,
(2) contacting the sample with one or more binding partners, each binding partner specific for a cellular or extracellular target,
(3) detecting binding partners bound to the sample by obtaining an image of the sample,
(4) clearing the binding partners from the sample, and
(5) repeating steps (2) through (4) one or more times.

In some embodiments, steps (2) through (4) are repeated at least 10 times, or at least 20 times.

In some embodiments, the sample is not substantially degraded throughout the method.

In some embodiments, the images obtained are overlayed and aligned to obtain a composite image.

In some embodiments, the sample is cleared using high temperature. In some embodiments, the sample is cleared using a temperature of about 80° C., optionally for 1-4 days.

In some embodiments, the sample is incubated with reducing agents. In some embodiments, the reducing agents are sodium sulfite and 1-thioglycerol.

In some embodiments, the binding partners are antibodies or antigen-binding antibody fragments.

In some embodiments, the targets are myelinated axons and fibers.

In some embodiments, the method further comprises detecting nucleic acids and/or lectins in the sample. In some embodiments, the sample is contacted with binding partners for DNA and lectin in addition to the binding partner for the target in step (2).

These and other aspects and embodiments of the invention will be described in greater detail herein.

### BRIEF DESCRIPTION OF DRAWINGS

FIGS. 1A-1K. Synchronizing Dialdehyde-tissue-gel Formation Enables Scalable Tissue Preservation (FIG. 1A) Chemical structures of various multifunctional fixatives. (FIG. 1B) Crosslinked protein gels before and after exposure to the elution condition. Scale bars, 10 mm. Polyacrylamide (AA) gel swelled and became fragile, whereas multifunctional fixative gels remained intact with minimal expansion. (FIG. 1C) Mass percent change of crosslinked protein gels after exposure to the harsh condition. EDGDE, GE21, and EX-313 were incapable of forming gels at low BSA concentration. Error bars show mean±SD. (FIG. 1D) The gelation time for protein gels crosslinked with GA is nearly 200-fold higher at pH 3 than it is at neutral pH at 4° C. Error bars show mean±SD. (FIG. 1E) Schematic diagram illustrating the process of scalable and uniform tissue-gel formation without perfusion using SWITCH. GA molecules diffuse into an intact tissue without reacting with biomolecules in pH 3 buffer (SWITCH-Off step). When GA is uniformly dispersed throughout the tissue, the sample is moved to pH 7 buffer (SWITCH-On step) to initiate global gelation/fixation and achieve uniform tissue preservation. (FIG. 1F) Coronal slices from the middle of whole rat brains passively fixed with (bottom) or without (top) SWITCH. After fixation, the middle coronal slices were cut and incubated in the elution condition for 1 hr. The core of the control slice completely disintegrated, whereas the SWITCH-processed slices remained intact. Scale bars, 6 mm. (FIG. 1G) Only ˜3% of proteins are lost in SWITCH-processed brain tissues as opposed to ˜10-30% with AA-based methods. Error bars show mean±SD. (FIG. 1H and FIG. 1I) Antigenicity of proteins is well preserved throughout the clearing process in SWITCH. Of the antibodies tested, 86 of 90 are compatible with SWITCH. (FIG. 1J and FIG. 1K) SWITCH-mediated fixation maximally preserves macroscopic (FIG. 1J) and microscopic (FIG. 1K) structures throughout the elution process. (FIG. 1J) Cross-sectional images of 1-mm-thick mouse coronal slices after exposure to the elution condition. The CLARITY-processed tissue shows significant tissue deformation and collapse, whereas the SWITCH-processed tissue is highly uniform with no signs of macroscopic deformation. Z-step size, 20 μm; 10×, 0.3 NA, water-immersion objective. Scale bars, 1 mm. (FIG. 1K) GFP-expressing neurons in the cortex of Thy-1-EGFP mouse brain before and after exposure to the elution condition and anti-GFP staining. 25×, 0.95 NA, water-immersion objective. Scale bars, 30 μm. See also Table 1.

FIGS. 2A-2H. SWITCH and Co-registration Algorithms Enable Highly Multiplexed Imaging at Single-cell Resolution. (FIG. 2A) The left image shows formalin-fixed postmortem human brain tissue (visual association cortex, Brodmann area 18). The right image shows a 100-μm section of this brain tissue after SWITCH processing. Scale bars, 5 mm (left), 300 μm (right). (FIG. 2B) Natural warping of the sample during imaging was enabled by mounting within a chamber space larger than the size of the sample. Representative cross-sections of the sample after several rounds of imaging are shown. Sample thickness, 100 μm. (FIG. 2C) Surface contour maps showing warping of the sample between imaging rounds. Attempted manual overlay of two PV datasets shows that sample warping is too severe for single-cell registration without computational correction. Scale bars, 50 μm. (FIG. 2D) A flow diagram depicting the sequence of events for automated co-registration of datasets. (FIG. 2E) Fully co-registered image showing an overlay of 9 rounds of immunostaining. A total of 22 rounds of staining of the same tissue was achieved. R#2 (Ibal), R#3 (GFAP), R#4 (calbindin, CB), R#5 (calretinin, CR), R#7 (PV), R#8 (Neuropeptide Y), R#9 (NeuN), R#18 (SMI-32), and R#19 (PV) were used for co-registration and subsequent quantitative analysis (see FIGS. 3A-30). The boxed regions indicate the ROI's shown in panels (FIG. 2F-FIG. 2H). Scale bar, 300 μm. (FIG. 2F) Vasculature labeling from 9 rounds of staining after co-registration. Scale bar, 200 μm. (FIG. 2G) PV cell counts between rounds 7 and 19. After 12 rounds of imaging, 99% of previously detected Pr cells were again detected and shown to overlay after co-registration of the datasets. (FIG. 2H) Images of individual channels with corresponding vasculature labeling. Scale bar, 50 μm. See also Table 2.

FIGS. 3A-3O. SWITCH Enables Proteomic Imaging and High-Dimensional Quantitative Phenotyping of Human Clinical Samples. (FIG. 3A) ROI from FIG. 2E showing semi-automatically detected locations and sizes of blood vessels (lectin) and diverse cell types (GFAP⁺, NeuN⁺, SMI-32⁺, CB⁺, CR⁺, PV⁺) in human visual cortex. The identified objects are overlaid on maximum intensity-projections of raw images of the corresponding channels (dark gray). Dashed lines divide cortical layers I-VI. (FIG. 3B) 3D rendering of the boxed region in (FIG. 3A) (200 μm wide×200 μm high×104 μm deep) showing identified cells and blood vessels. (FIG. 3C) A heat map of the soma size distribution of NeuN⁺ cells, showing bimodal peaks at cortical layers III and V. (FIG. 3D) Density profiles of various cell types. (FIG. 3E) Comparison of cell sizes among different types of cells. One-way ANOVA was performed (***P<0.001; N=1,176, 7,835, 249, 1,044, 364 and 449 for each column). Post hoc tests were mostly P<0.001 except for three non-significant (n.s.) cases. (FIG. 3F) Distribution of neurons expressing various subsets of calcium-binding proteins in the human visual cortex. Raw images in the middle columns show CB⁺/CR⁺ or CB⁺/PV⁺ neurons (arrows). (FIG. 3G) A representative NeuN⁺/SMI-32⁺/CB⁺/PV⁺ cell. (FIG. 3H) Cell counts and densities in different cortical layers. Cortical layers with the highest density for each neuronal channel arc highlighted. (FIG. 3I) Cell densities for combinatorial co-expression of three interneuronal markers. (FIG. 3J) Statistics for NeuN⁻ neurons. (FIG. 3K) Representative images showing NeuN⁻/CB⁺, NeuN⁻/CR⁺, and NeuN⁻/PV⁺ cells (arrows). The arrowhead indicates a CR⁺ cell with low NeuN immunoreactivity. (FIG. 3L) Comparison of cell-to-nearest vessel distances along cortical depth as measured from cell centroids to vascular boundaries. Post hoc tests following one-way ANOVA (P<0.001; N=935, 4,101, 210, 817, 265 and 331 for each column) were mostly n.s. except for three cases displayed. *P<0.05. (FIG. 3M) Vascular density and distance-to-nearest vessel profiles of GFAP⁺ or NeuN⁺ cells along cortical depth. Mean distances from NeuN⁺ (D_{N}) and GFAP⁺ (D_{G}) cells and all extravascular pixels (Dₚ) are calculated and plotted. Diagrams illustrate the calculation of the three distances. (FIG. 3N) Cell-to-nearest vessel distances from NeuN⁺ cells in two regions—a (N=570) and b (N=445) in (M)—before (D_{N}) and after (D_{N}-Dₚ) correction. (FIG. 3O) Distribution profile of extravascular pixel- or cell-to-nearest vessel distances showing similar patterns. Three interneuronal markers are plotted together. Error bars are shown with mean±SEM. Scale bars, 200 μm (FIG. 3A), 50 μm (FIGS. 3F, 3G, and 3K). See also Table 3.

FIGS. 4A-4F. SWITCH Enables Simple, Rapid, and Scalable Tissue Clearing. (FIG. 4A) Images of 1-mm coronal blocks of an adult mouse brain hemisphere before and after clearing at 37° C. for 24 hr or 80° C. for 12 hr. The lipid-extracted tissues were refractive index (RI)-matched. Scale bars, 3 mm. (FIG. 4B) Images of mouse brain hemispheres lipid-extracted at 80° C. for 10 days with 200 mM SDS containing 0-50 mM sodium sulfite (SS) as an anti-browning agent. Note that the tissues were not RI-matched. Scale bars, 6 mm. (FIG. 4C) Images of intact adult mouse brains cleared at 37° C. (top panel) and 60° C. (middle panel) and 80° C. (bottom panel) with and without 1-thioglycerol (TG). Browning in high-temperature clearing was effectively prevented by TG. Scale bars, 3 mm. (FIG. 4D) High-temperature (80° C.) clearing of whole rat brain with and without TG. Scale bars, 6 mm. (FIG. 4E) Clearing of human and marmoset samples at 80° C. Scale bars, 6 mm. (FIG. 4F) Rapid clearing of various organs at 80° C. with and without 50 mM SS. Cleared rat spinal cord is not RI-matched. Scale bars, 3 mm.

FIGS. 5A-5K. SWITCH Enables Visualization and Quantitative Analysis of Entire Myelinated Fiber Tracts. (FIG. 5A) DiD and MBP staining on a SWITCH-processed mouse brain slice showing complete overlap between DiD and MBP. Scale bar, 10 μm. (FIG. 5B) DiD staining with PBST or with PBS+10 mM SDS buffer. DiD staining is completely inhibited in PBS+10 mM SDS buffer. Green, sytol6; red, DiD; scale bars can be seen in the color version of the drawings, 100 μm (top, bottom panels), 10 μm (middle panel). (FIG. 5C) DiD staining of a 1-mm-thick mouse coronal block using PBST for 1.5 days at 37° C. Only tissue surface is labeled. Scale bar, 200 μm. (FIG. 5D) DiD staining of a 1-mm-thick mouse coronal block using SWITCH. The sample was first incubated in DiD, 10 mM SDS containing PBS buffer for 24 hr, then moved to PBST and incubated for 0.5 day at 37° C. The whole sample is uniformly labeled. Scale bar, 200 μm. (FIG. 5E) Volume image of a 1-mm-thick mouse brain coronal slice stained with DiD to visualize myelinated tracts acquired using a confocal microscope. The volume contains both the striatum and the cortex. Scale bar, 200 μm. (FIG. 5F) Maximum intensity projection of the subvolume (illustrated in white in the volume image in FIG. 5E) shows fascicles from the striatum diverging at the corpus callosum and fibers near that area in the cortex forming a grid pattern. Scale bar, 200 μm. (FIG. 5G) Enlarged images of the selected regions of interest in (FIG. 5F) shows the fibers in the cortex arranged in a grid pattern. Fibers are colorized based on orientation. Scale bar, 100 μm. (FIG. 5H) Analysis of all the fibers in the entire volume shows that most fibers make an 89° intersection in xy and yz and an 88° intersection in xz. (FIG. 5I) Analysis of all the fascicles in the entire volume shows that they make an 87° turn in xy, a 26° turn in yz, and a 30° turn in xz. (FIG. 5J) Volume image of a mouse brain hemisphere stained with DiD to visualize myelinated tracts acquired using a custom-built light-sheet microscope. Scale bar, 1 mm. (FIG. 5K) Representative images showing individual fibers and fascicles in three different brain regions in (FIG. 5J). Str, striatum; Hipp, hippocampus; TH, thalamus. Scale bars, 200 μm.

FIGS. 6A-6C. SWITCH Increases Uniformity of Antibody Labeling in Thick Tissues. (FIG. 6A) Antibody staining of cleared 100-pm mouse brain sections in PBST and various concentrations of SDS in PBS. SDS effectively inhibits antibody-antigen binding in a concentration-dependent manner. Scale bar, 200 μm. (FIG. 6B and FIG. 6C) Histone H3 staining of 1-mm-thick mouse cerebral cortex blocks in PBST (FIG. 6B) and using SWITCH (FIG. 6C). Control sample was incubated in antibody-containing PBST for 12 hr then washed for 12 hr. SWITCH sample was incubated in antibody-containing SWITCH-Off solution for 12 hr then washed in SWITCH-On solution for 12 hr. Sections from the top, middle, and bottom of the blocks are shown. 3D renderings were generated from the ROIs shown. SWITCH sample showed vast increase in uniformity of labeling compared to control. Scale bars, 150 μm (FIG. 6B, left panel), 200 μm (others).

FIG. 7. Overview of various rounds of system-wide control of interaction time and kinetics of chemicals (SWITCH).

### DETAILED DESCRIPTION OF INVENTION

The invention provides, inter alia, a method for preserving samples including but not limited to animal and human tissue samples. The samples so preserved are rendered sufficiently mechanically and chemically stable for multiplexed imaging. Generally, the method involves a first step of permeating a crosslinking agent (or crosslinker, as the terms arc used interchangeably herein) throughout the entire sample in a condition that reduces crosslinking activity to a negligible or near negligible level. In some instances, such first condition minimally comprises a low pH, including a pH of about 3. The sample may be contacted with the crosslinking agent for a period of time sufficient for the crosslinking agent to permeate the sample. This period of time may be minutes, hours, or days, and may depend upon the size, dimensions and/or mass of the sample, as well as the nature of the sample. The Examples demonstrate one exemplary processing in which a post-mortem human tissue sample was processed by incubating the tissue at about 4° C. for about 2 days.

The method then further involves changing the condition such that crosslinking activity occurs. In some instances, this second condition minimally comprises a neutral pH, including a pH of about 6.5-7. The sample may be incubated at this second condition for a period of time sufficient for the crosslinking agent to crosslink targets (typically biomolecules) in the sample. This period of time may be minutes, hours, or days, and similarly may depend upon the size, dimensions and/or mass of the sample, as well as the nature of the sample. In the exemplary process described in the Examples, the tissue sample was incubated with PBS for 2-3 days at 4° C. and then 2-7 hours at 37° C.

In other instances, the pH is increased from the first condition to the second condition such that the first condition pH is lower than the second condition pH. The first condition pH may be in the range of about 6.5 to about 8, including about 7 to about 8, provided it is lower than the second condition pH. The second condition pH may be in the range of about 8 to about 11 including about 8.5 to about 11, about 9 to about 11, about 9.5 to about 11, and about 10 to about 11, provided it is higher than the first condition pH. The second condition pH may be about 1, or about 2, or about 3, or more pH units more than the first condition pH.

As will be clear, both the first and the second incubation steps include the presence of crosslinking agent. The first step may comprise a higher concentration of the crosslinking agent than the second step. In the exemplary protocol in the Examples, the crosslinking agent was present at about 4-10% in the first step and then at about 1-4% in the second step.

Crosslinking of the sample as occurs in the second step may be facilitated by an increase in temperature as well. Thus, the first step (the “permeation” step) may be performed at a low temperature such as at about 4° C. and the second step (the “crosslinking” step) may be performed at a higher temperature such as 37° C.

The sample may or may not have undergone some degree of fixation depending on the embodiment. Such pre-fixation may include paraformaldehyde fixation. The method however may be performed without pre-fixation.

### SWITCH-Mediated Sample Preservation

The preservation method comprises contacting the sample with a crosslinker such as but not limited to a multifunctional crosslinker for a period of time sufficient to allow the crosslinker to uniformly disperse throughout the entire sample under a condition(s) that renders the crosslinker relatively inactive, then activating the crosslinker by changing the condition to one that renders the crosslinker active or that increases the activity of the crosslinker.

As used herein, a condition that renders a crosslinker inactive or relatively inactive in the first step of the preservation method intends that the condition slows down the crosslinking reaction. It does not intend that the crosslinker is irreversibly inactivated. Rather, its activity is reduced to limit or prevent any crosslinking during that first permeation step. As described in greater detail herein, the pH affects the amines that are targeted by the crosslinker, rendering them either suitable to attack by the crosslinker or resistant from such attack.

Once the sample is sufficiently crosslinked, any remaining active crosslinker may he irreversibly inactivated. This may be accomplished in a number of ways depending in part on the type of crosslinker used. In some embodiments that use glutaraldehyde (GA) as the crosslinker, GA may be inactivated by incubation of the sample with a solution containing glycine and acetamide. In some embodiments, this incubation may occur for about 24 hours at 37° C. The sample may then be washed in neutral pH buffer (e.g., PBS) in order to wash away unreacted substituents and the like.

Crosslinkers suitable for use in the methods provided herein typically are chemical compounds having at least two reactive moieties that are capable of binding, preferably irreversibly, to at least two sites in the sample. These sites may be on the same target or on different targets. Preferably, they are on different targets in the sample. The crosslinker may be bifunctional (i.e., having two reactive moieties) or multifunctional (i.e., having more than two reactive moieties, including 3, 4, 5, or more such moieties). The reactive moieties may be identical or they may be different.

In some embodiments, the crosslinkers are capable of rapid penetration without the use of perfusion and have a high degree of molecular crosslinking as this latter feature improves sample durability. Suitable crosslinkers may also be small and have high water solubility. In some embodiments, crosslinkers include but are not limited to ethylene glycol diglycidyl ether (EGDGE), dipropylene glycol diglycidyl ether (GE23), 1,4-butanediol diglycidyl ether (GE21), glycerol polyglycidyl ether (EX-313), and glutaraldehyde (GA). In some embodiments, the crosslinking agent is ethylene glycol diglycidyl ether (EGDGE). In some embodiments, the crosslinking agent is 1,4-butanediol diglycidyl ether (GE21). In some embodiments, the crosslinking agent is glycerol polyglycidyl ether (EX-313). In some embodiments, the crosslinking agent is glutaraldehyde (GA). In some embodiments, the crosslinking agent is polyglycerol-3-polyglycidyl ether (GE38).

Other multifunctional crosslinking agents that may be used in the methods provided herein include aldehydes, e.g., succinaldehyde, oclanedialdehyde, and glyoxal; halo-triazines, e.g., cyanuric chloride; halo-pyrimidines, e.g., 2,4,6-trichloro/bromo-pyrimidine; anhydrides or halides of aliphatic or aromatic mono- or di-carboxylic acids, e.g., maleic anhydride, (meth)acryloyl chloride, chloroacetyl chloride; N-methylol compounds, e.g., N-methylol-chloro acetamide; di-isocyanates or di-isothiocyanates, e.g., phenylene-1,4-di-isocyanate, aziridines; and epoxides, e.g., di-epoxides, tri-epoxides, and tetra-epoxides.

In some embodiments, the first step of the preservation method is carried out at a low pH or at an acidic pH. Such low pH or acidic pH includes a pH range of about 2 to about 5, about 2.5 to about 4.5, about 3 to about 4, about 2.5 to about 3.5, or about 3. In some embodiments, the low pH or acidic pH is a pH of 5 or less, and may range from about 2 to about 5, about 3 to about 5, or about 4 to about 5. In some embodiments, the low pH or acidic pH is a pH of 4 or less, and may range from about 2 to about 4, or about 3 to about 4.

In some embodiments, the second step of the preservation method is carried out at a neutral pH. Such neutral pH includes a pH range of greater than 5, including about 5.5 to about 9, about 5.5 to about 8.5, about 6 to about 8, about 6.5 to about 7.5, and about 7. In some embodiments, the pH is in a range of about 6 to about 7, including about 6.5 to about 7, including about 6.8.

In some embodiments, the first step is carried out at a pH in the range of about 6 to about 8 or about 7 to about 8 and the second step is carried out at a pH in the range of about 8 to about 11 or about 9 to about 11 or about 9 to about 10, provided the pH of the first step is lower than the pH of the second step.

The term about as used in the context of pH values intends a variation in the range of +/−0.5, including +/−0.4, +/−0.3, +/−0.2, and +/−0.1.

The conditions in the first step collectively will result in a reduced or nearly negligible crosslinking activity. Those conditions collectively may reduce crosslinking activity to about 10%, 1%, 0.1%, 0.01%, or 0.001% of the crosslinking activity that occurs at for example the conditions in the second step of the method (e.g., at neutral pH, and optionally at 37° C.).

The amount of crosslinker used may vary, as discussed above. It may range from 1-50%, 1-40%, 1-30%, 1-25%, 1-20%, 1-15%, 1-10%, and 1-5%. In the first step, a higher concentration of the crosslinker may be used, including for example in the range of 4-10% or 5-10%. In the second step, a lower concentration of the crosslinker may be used, including for example in the range of 1-5% or 1-4%.

### Sample Selection

The methods may be used to preserve and image a variety of samples including but not limited to animal and human tissue samples. In some embodiments, samples are derived from humans, companion animals such as dogs or cats, agricultural animals such as cows, sheep and pigs, rodents such as rats or mice, zoo animals, primates such as monkeys, and the like. The samples may be from a variety of organ and non-organ tissues, including but not limited to brain, lung, liver, kidney, spinal cord, etc. The Examples demonstrate preservation and multiplexed imaging of rat, human, and marmoset brains; rodent lung, kidney, heart, and liver; and spinal cords.

### Sample Imaging

Once the sample is preserved according to the methods provided herein, it may be analyzed for the presence of one or more targets such as proteins and other biomolecules. The presence of the one or more targets may be accomplished by contacting the sample with binding partners for the targets of interest. Such binding partners may be applied to the sample consecutively or simultaneously. Importantly, samples preserved according to the methods of this disclosure are able to undergo multiple rounds of binding partner labeling, imaging, and binding partner removal (or destruction) without significant or any appreciable effect on tissue architecture or target antigenicity.

The binding partners may be any molecule or compound capable of binding, preferably specifically, to the target of interest. It should also be possible to remove the binding partner (or destroy the binding partner) in order to facilitate successive rounds of labeling and imaging of the sample. Binding partners may be, without limitation, amino acid based or nucleic acid based. An example of amino acid based binding partners is antibodies and antigen-binding antibody fragments. The antibodies and fragments thereof may be monoclonal antibodies. Another example is a peptide aptamer. An example of nucleic acid based binding partners is aptamers.

As used herein, “antibody” includes full-length antibodies and any antigen binding fragment (e.g., “antigen-binding portion”) or single chain thereof. The term “antibody” includes, without limitation, a glycoprotein comprising at least two heavy (H) chains and two light (L) chains inter-connected by disulfide bonds, or an antigen binding portion thereof. Antibodies may be polyclonal or monoclonal; xenogeneic, allogeneic, or syngeneic; or modified forms thereof (e.g., humanized, chimeric).

As used herein, “antigen-binding portion” of an antibody, refers to one or more fragments of an antibody that retain the ability to specifically bind to an antigen. The antigen-binding function of an antibody can he performed by fragments of a full-length antibody. Examples of binding fragments encompassed within the term “antigen-binding portion” of an antibody include (i) a Fab fragment, a monovalent fragment consisting of the VH, VL, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VH and VL domains of a single arm of an antibody, (v) a dAb fragment (Ward et al., Nature 341:544 546, 1989), which consists of a VH domain; and (vi) an isolated complementarity determining region (CDR) or (vii) a combination of two or more isolated CDRs, which may optionally be joined by a synthetic linker.

Furthermore, although the two domains of the Fv fragment, VH and VL, are coded for by separate genes, they can be joined, using recombinant methods, by a synthetic linker that enables them to be made as a single protein chain in which the VH and VL regions pair to form monovalent molecules (known as single chain Fv (scFv); see, e.g., Bird et al. Science 242:423 426, 1988; and Huston et al. Proc. Natl. Acad. Sci. USA 85:5879-5883, 1988). Such single chain antibodies are also encompassed within the term “antigen-binding portion” of an antibody. These antibody fragments arc obtained using conventional techniques known to those with skill in the art, and the fragments are screened for utility in the same manner as are intact antibodies.

As used herein, “peptide aptamer” refers to a molecule with a variable peptide sequence inserted into a constant scaffold protein (see, e.g., Baines I C, et al. Drug Discov. Today 11:334-341, 2006).

As used herein, “nucleic acid aptamer” refers to a small RNA or DNA molecules that can form secondary and tertiary structures capable of specifically binding proteins or other cellular targets (see, e.g., Ni X, et al. Curr Med Chem. 18(27): 4206-4214, 2011).

Typically, these binding partner will themselves he labeled with detectable labels. The detectable labels may be fluorophores. Examples of fluorophores include, without limitation, xanthene derivatives (e.g., fluorescein, rhodamine, Oregon green, eosin and Texas red), cyanine derivatives (e.g., cyanine, indocarbocyanine, oxacarbocyanine, thiacarbocyanine and merocyanine), naphthalene derivatives (e.g., dansyl and prodan derivatives), coumarin derivatives, oxadiazole derivatives (e.g., pyridyloxazole, nitrobenzoxadiazole and benzoxadiazole), pyrene derivatives (e.g., cascade blue), oxazine derivatives (e.g., Nile red,

Nile blue, cresyl violet and oxazine 170), acridine derivatives (e.g., proflavin, acridine orange and acridine yellow), arylmethine derivatives (e.g., auramine, crystal violet and malachite green), and tetrapyrrole derivatives (e.g., porphin, phthalocyanine and bilirubin). Other detectable labels may be used in accordance with the present disclosure, such as, for example, gold nanoparticles or other detectable particles or moieties.

When two or more binding partners are used simultaneously, it may be preferable to label such binding partners with labels that are spectrally distinct (i.e., that can be distinguished from other label being used simultaneously). In other words, in some embodiments, the labels can be distinguished from each other.

Virtually any target of interest may be imaged using the methods provided herein provided a suitable binding partner exists. Examples of targets are provided in Table 1. The targets may be found in various locations including without limitation at the membrane (e.g., membrane bound), in the cytoplasm, in various organelles such as but not limited to the nucleus, and/or at synapses, and the like.

The following Examples are included for purposes of illustration and are not intended to limit the scope of the invention.

### EXAMPLES

### Materials and Methods

Mice

Young adult male and female C57BL/6 and Thy1-eGFP-M mice were housed in a reverse 12-hr light/dark cycle with unrestricted access to food and water. All experimental protocols were approved by the MIT Institutional Animal Care and Use Committee and Division of Comparative Medicine and were in accordance with guidelines from the National Institute of Health.

SWITCH—Mediated Tissue Preservation

PFA-fixed human samples were washed in a solution consisting of 50% PBS titrated to pH 3 using HCl, 25% 0.1 M HCl, and 25% 0.1 M potassium hydrogen phthalate (KHP). This wash solution was then replaced with fresh solution with the addition of 4-10% GA. The samples were then incubated in this pH 3 solution at 4° C. for 2 days with gentle shaking. The acidic pH of this solution greatly slows down the reaction speed of aldehyde fixatives. The solution was then replaced with PBS with the addition of 1-4% GA and the sample was again allowed to incubate for 2 days at 4° C. and 2-7 hr at 37° C. with gentle shaking. The sample was then washed in PBS at room temperature (RT) for 1 day with gentle shaking. After washing, reactive GA within the sample was inactivated by incubation in a solution consisting of 4% glycine and 4% acetamide for 1 day at 37° C. with gentle shaking. Finally, the sample was washed for 1 day in PBS at RT with gentle shaking.

Passive Clearing with Thermal Energy

Aqueous clearing solution containing 200 mM SDS, 10 mM lithium hydroxide, 40 mM boric acid, and a variable amount of anti-browning agent (i.e., 0-50 mM sodium sulfite or 0-0.5% [w/v] 1-thioglycerol) was titrated to pH 9 using sodium hydroxide before use. Samples were incubated at 60-80° C. until clear using Easy-Passive (EP-1001; Live Cell Instrument) or a water bath. For high temperature clearing, samples were incubated in 40 mL of clearing solution for 8 hr at RT with gentle shaking to allow for the anti-browning agent to diffuse throughout the tissue. Samples were then transferred to a water bath set at 60-80° C. The clearing buffer was replaced if any noticeable color was observed in solution at any point during clearing. To remove the remaining anti-browning agent and SDS after clearing, the samples were washed at 37° C. for 24 hr in 40 mL PBST containing 0.02% sodium azide as a preservative.

Sample Delabeling

Imaged samples were delabeled in clearing solution at 60-80° C. (elution condition) for 1-2 days for large samples and 0/N for thin samples. Sulfites were added for large samples to prevent browning during the extended delabeling process.

SWITCH-Mediated Fluorescent Labeling

Samples were incubated in SWITCH-Off solution (0.5 or 10 mM SDS in PBS) 0/N with gentle shaking at 37° C. and transferred to a fresh volume of SWITCH-Off solution (containing molecular probes) just enough to cover the sample. Samples were incubated at 37° C. with gentle shaking and times were scaled with sample size. Samples were then transferred to a large volume of PBST (SWITCH-On) and incubated at 37° C. with gentle shaking.

Perfusion

Mice were transcardially perfused with ice-cold PBS and a solution consisting of 4% PFA and 1-4% GA in PBS. Brain tissues were harvested and incubated in the same fixative solution at 4° C. for 2-3 days and 2-7 hr at 37° C. with gentle shaking to allow for uniform fixation throughout the sample. This incubation time is critical for mitigating the effects of variable perfusion quality and promotes uniform structural and molecular preservation of the sample throughout the SWITCH process, as samples that are not ideally preserved will experience greater loss of biomolecules and a greater degree of sample deformation.

GA Gelation Time Experiment

All reagents and containers were first cooled to 4° C. and handled on ice. For pH 7 gels, 10 mL of a PBS solution containing 10% BSA was made prior to GA injection. For pH 3, 10 mL of a 0.1 M KHP buffer was titrated to pH 3 with HCl. Once the pH 3 and 7 BSA solutions were prepared, GA was added to a final concentration of 4% and a timer was started. Gelation was judged by inverting the tube and inspecting for fluid flow. The time required to form rigid gels was recorded in 3 replicates of the pH 3 and 7 condition.

Gel Denaturation Experiment

For acrylamide (AA) gels, 10 mL of a 4% PFA, 5-15% bovine serum albumin (BSA), 4% AA, and 0.25% VA-044 solution was prepared in PBS. The polymerization was carried out under vacuum for 2 hr at 37° C. in a 15 mL tube. For GA gels, 10 mL of a 5-15% BSA and 1% GA solution was prepared in PBS and allowed to gel at room temperature for 2 hr in a 15 mL tube. For the epoxide gels, 10 mL of a 5-15% BSA and 15% epoxide (i.e., EX-313, GE31, GE22) solution was prepared in 0.1 M carbonate buffer at pH 9 and allowed to gel at 37° C. for 8 hr in a 15 mL tube. Each gel was extracted from the tubes and cut into approximately 5-mm-thick disks. The disks were then washed overnight in PBST to remove any unreacted reagents. After washing, each disk was massed and photographed. The disks were then transferred to an 80° C. water bath and incubated overnight. The disks were removed, massed, and photographed again.

Sodium Borohydride Treatment

Sodium borohydride (SB) buffer was made immediately before use by making a 1 mg/ml SB solution with PBS. For 100-μm sections, the tissue was treated 3 times, 10 minutes each. For whole brain, the tissue was treated 3 times, 3 hr each. For both, incubation was done at room temperature without shaking

mRNA FISH

Mice were perfused with ice-cold PBS and then with fixative (4% PFA and 1% GA in PBS). Brains were incubated in the fixative for one day at 4° C. and then 6 hr at RT for post-fixation. Coronal sections were prepared with a vibratome and sections were inactivated at RT for 6 hr, followed by tissue clearing under 37° C. or 70° C. All solutions were prepared by using diethylpyrocarbonate (DEPC)-treated water. Digoxigenin- and 2,4-dinitrophenol (DNP)-labeled fos cRNA probes were detected using horseradish peroxidase-conjugated antibodies. FISH signals were visualized using a tyramide amplification kit (Perkin Elmer).

Refractive Index Matching

A customized refractive index (RI)-matching solution was made by dissolving 50 g diatrizoic acid, 40 g n-methyl-d-glucamine, and 55 g iodixanol per 100 mL water. Cleared samples were incubated in 10 mL of this solution at RT with gentle shaking for 2 days prior to imaging, replacing the solution after the first day. The listed components and their proportions were chosen to adjust the pH and RI for ideal optical clearing (basic pH with RI near 1.47) as well as optimize the osmolarity of the solution to reverse the sample expansion observed after clearing. The contrast agents, diatrizoic acid and iodixanol, significantly affect the RI of the solution, while n-methyl-d-glucamine is used to adjust the pH to more basic values. All components were considered when optimizing for osmolarity. RI was measured using an Abbemat WR/MW automatic multiwavelength refractometer.

Mounting and Imaging

To facilitate the use of long working distance immersion objectives, samples were mounted between a slide glass and a glass-bottom Willco dish. Blu-Tack adhesive was rolled into a cylindrical shape of a thickness slightly greater than that of the sample and was placed in a circular orientation on the slide glass with a small opening to allow addition of immersion medium after chamber construction. The sample was placed within the Blu-Tack circle and the Willco dish was secured onto the adhesive, pressing just firmly enough to make slight contact with the sample. This contact prevents the sample from moving during the imaging process, but minimizes sample deformation. For multiplexed staining experiments, contact was not made with the sample Taking care to avoid introduction of bubbles, RI-matching solution was injected to fill the void space, and the opening was then closed using fast-curing epoxy glue. Three microscope systems were used for the experiments in this study:

Olympus two-photon microscope system (FV1200MPE) equipped with a 25× CLARITY-optimized objective (prototype; NA, 1.0; WD, 8.0 mm), a 10× CLARITY-optimized objective (XLPLN10XSVMP; NA, 0.6; WD, 8.0 mm), a 10× water-immersion objective (NA, 0.30; WD, 3.6 mm), and a 40× oil-immersion objective (UPLSAPO40XS; NA, 1.25; WD, 0.3 mm). 405, 488, and 635 nm 1p lasers were used;

Leica TCS SP8 microscope system equipped with a white-light laser, spectral detection system, a 20× water-immersion objective (NA, 0.50; WD, 3.5 mm) and a 25× water-immersion objective (NA, 0.95; WD, 2.4 mm). Custom-made light-sheet microscope equipped with 10× CLARITY-optimized objective (modified from Tomer et al., 2012). Sample datasets were visualized with IMARIS (Bitplane).

Protein Loss Assay

Mouse brain samples were prepared using various preservation methods, hemisected, and then cut into 1-mm sections. The collection of sections from each hemisphere was massed and then placed into 5 mL of 200 mM SDS clearing solution. The samples were incubated at 37° C. with gentle shaking for 2 weeks. A small aliquot was taken from each tube and analyzed using the Bio-Rad DC protein assay kit to quantify the degree of protein loss from the samples.

Microstructure Preservation Assay

A Thy1-eGFP-M mouse was perfused with SWITCH fixative solution and the sample was cut into 1-mm sections. The eGFP expression on the surface of a section was imaged using confocal microscopy and then sample was then subjected to clearing under harsh conditions (200 mM SDS, 80° C.) for 1 day. The sample was then labeled using anti-eGFP antibodies and the same region was imaged again using confocal microscopy.

Macrostructure Preservation Assay

Samples processed using SWITCH and CLARITY were cut in 1-mm sections and subjected to clearing under harsh conditions for 1 day. The samples were then mounted in a chamber larger than the size of the sample to prevent compression and imaged using confocal microscopy. RI-matching solution was used to facilitate imaging of the sample. Cross-sections from the samples were visualized using IMARIS.

Human Tissue Samples

Samples of fixed autopsy tissue were obtained from the Neuropathology Core of the Massachusetts Alzheimer Disease Research Center. Tissue was collected and banked in accordance with approval from the local Institutional Review Board. All samples studied came from subjects without evidence of neurologic disease on clinical grounds at the time of death and without evidence of significant disease processes upon full neuropathologic examination.

Multiplexed Labeling of Thin Samples

A human clinical sample containing visual cortex was obtained and processed using SWITCH. After fixation, 100-μm sections were obtained from the sample and cleared under harsh conditions. In each round, the sample was labeled with DAPI, DyLight 488-conjugated lectin, and a variable antibody using standard immunolabeling procedures. The sample was mounted in a chamber larger than the size of the sample, imaged using confocal microscopy, and then delabeled under harsh conditions 0/N. RI-matching solution was used to facilitate imaging of the sample.

Co-Registration of Multiplexed SWITCH Experiments

To register the set of SWITCH experiments from a single tissue, one of the experimental rounds for a tissue is arbitrarily chosen as the fixed reference to which the rest of the experiments will be registered. Each experiment has one fluorescence channel dedicated to lectin, which allows the software to identify distinctive points in the vasculature in order to achieve the fine morphological adjustments across the tissue volume. We used a 3D Harris Corner detector (Harris and Stephens, 1988) to find those keypoints and a 31) modification of the SIFT descriptor (Lowe, 2004; Sco anner et al., 2007) to calculate correspondences. For robustness, we instituted a variation of RANSAC (Fischler and Bolles. 1981) to test affine transformations on local subvolumes, confirming that keypoint correspondences between experiments were legitimate. Finally, with the validated keypoints, a thin plate spline interpolation (Bookstein, 1989) was implemented to warp the tissue in a physically plausible manner.

Each experiment was processed individually by eye before using software to calculate the registration: The size of the interrogated tissues, approximately 1 mm³ in volume with 1.09 μm×1.09 μm×1.99 μm resolution, required imaging subvolumes that were stitched together using the Leica or Olympus microscope software. The resulting tissue volume, a 4-dimensional object for the three spatial coordinates and the fluorescence channel, was examined by human eye using FIJI (Schindelin et al., 2012) to ensure image quality and a common orientation across experiments. Additionally, a rectangular crop was made around the tissue to remove unnecessary, blank voxels. Each individual experiment was then processed using a MATLAB computational pipeline developed for SWITCH and shared online via Github (see “github” website, identified as “dgoodwin208”, then “Registration”).

The registration pipeline has five primary steps based on the Lectin channel of each experiment. First, the image volume is partitioned into 25 subvolumes for parallelization of work and robustness checking in later steps. Each subvolume then identifies distinguishable keypoints using a 3D Harris Corner Detector and uses a 3D modification of SIFT written by Scovanner (cs.ucf.edu/˜pscovann/) to create a descriptor vector associated with the keypoint. Note that we calculated keypoints and descriptors at multiple scale levels, achieved by convolution with Gaussian kernels of progressive size, to ensure a sufficient degree of scale invariance to the detected descriptors to successfully find matches despite differences in microscope setups across experiments. The calculations for keypoints and descriptors was often calculated in parallel across subvolumes to save time.

The third step is that each subvolume searches for corresponding keypoints in the appropriate subvolume in the reference experiment using the SIFT metric for measuring similarity between descriptors. To accomplish this we used the open-source VLFeat (available at the vlfeat website) implementation of SIFT matching algorithm. The fourth step is that the keypoint correspondences are validated via calculating affine transformations of random subsets of 4 corresponding keypoints, and the number of inliers of the resulting transformation assessed by a voxel distance threshold of 3 pixels. Each time a pair of corresponding points is counted as an inlier, it receives a vote, and after the order of 10⁶ affine transformations, correspondence pairs with at least 80% of the votes of the highest voted pair are kept as legitimate correspondences. Finally, the validated correspondences are used to calculate a thin plate spline for the entire volume using an open-source TPS implementation written by Yang, Foong and Ong (available at the mathworks website, see matlabcentral/fileexchange/47409-glmdtps-registration-method/content/GLMD_Demo/src/TPS3D.m), resulting in a highly accurate warp to match the morphology of the reference experiment.

Semi-Automatic Identification of Cells and Blood Vessels

Image volumes were displayed and analyzed using custom-built graphical user interface software developed with Delphi XE4 (Embarcadero Technologies). Each image section was preprocessed to correct the inhomogeneous illumination at each image tile. In detail, we subtracted the mean intensity of a 100×100 μm² window centered to each pixel from its intensity to uniformize the background intensity. A different algorithm was devised specifically for each marker to semi-automatically detect the centroid location and soma size of all cellular objects and vascular pixels. In general, a spherical soma volume was isolated according to the best contrast between intrasomal pixels and background pixels by increasing the size of concentric spheres, and the soma size was determined as the spherical diameter. After this automatic detection process, we corrected misidentified cell bodies manually, and the portion of the correction was less than 10%. We applied normalization of foreground signal and a Gaussian filter to the NeuN channel prior to the analysis. SMI-32⁺ cells were fully recognized manually according to their characteristic feature of the soma connected to a vertically oriented fiber with a large nuclear shadow, and the determination of their coordinates and soma sizes was aided by an automation module of the software. Each section in the lectin channel was converted to a vascular pixel mask image according to a customized threshold, and unconnected small clusters of pixels were removed.

Quantitative Analysis of the Co-registered Image Channels

We used a series of custom-built software developed with Delphi XE4 for the quantitative analysis. For co-expression analysis of two or more markers, an initial decision was made by checking whether the centroids of cells in each channel fell within a 5-μm distance. This classification was then manually verified with a quick review software tool. Especially, NeuN⁻ neurons were carefully reviewed, and any weak NeuN signal that changed synchronously with the other marker signal was identified as NeuN⁺. Cell density, vascular density, and cell- or pixel-to-vessel distance along cortical depth were obtained from their average of 50- or 100-μm window with a 50- or 100-μm interval, and data points of less than 10 objects were excluded from plotting. Cell and vascular densities were corrected to exclude the dead volume outside the tissue in the ROI. The cell-to-vessel distance was calculated as a distance from the centroid location of the cell to the nearest vascular pixel. Cells or pixels positioned at the sections containing any incomplete vascular information (z <24 μm or z≥80 pm) were excluded from the analysis of distance to nearest vessel. The distribution profile of cell-to-vessel distance was obtained with a 3-μm interval.

SWITCH-Mediated Myelinated Fiber Labeling

To create a DiD solution for myelinated fiber labeling, 1 mg of DiD powder was dissolved in 200 μL of a solution consisting of 10 mM SDS in PBS (SWITCH-Off). For 1-mm mouse sections, samples were incubated in SWITCH-Off solution O/N with gentle shaking at 37° C. The solution was replaced with a volume of fresh SWITCH-Off buffer that was sufficient to cover the sample, and 1 μL of the DiD solution was added. The sample was allowed to incubate for 1 day at 37° C. with gentle shaking, at which point the sample was moved to a large volume of PBST (SWITCH-On) for 1 day at 37° C. with gentle shaking. The sample was imaged using confocal microscopy. RI-matching solution was used to facilitate imaging of the sample.

For mouse hemispheres, the sample was incubated in SWITCH-Off solution at 37° C. O/N with gentle shaking and then transferred to a volume of fresh SWITCH-Off solution sufficient to cover the sample, at which point 2 μL of the DiD solution was added. The sample was incubated in this solution at 37° C. for 4 days with gentle shaking and then moved to a large volume of SWITCH-On solution for 1 day at 37° C. with gentle shaking. RI-matching solution was used to facilitate imaging of the sample.

Orientation Analysis of Myelinated Fibers

Analysis was performed on planar images in the xy, yz, and xz planes using OrientationJ (available at the big www website, see epfl.ch/demo/orientation/). Specifically, OrientationJ was used to calculate the preferred orientation of each pixel (ranging from −90° to) 90° using the corresponding finite difference gradient. This generates planar images whose pixel values correspond to the angular component in that plane (i.e., xy planar image contains θ_{xy}). After obtaining this orientational information in xy, yz, and xz for all slices, the separate components of the orientation (i.e., θ_{xy} contains x and y components of the orientation) are added together to yield three-dimensional orientation vectors. The orientation vectors represent the orientations of the fibers and the fascicles. These orientation vectors can be binned according to their angles to yield information about how the fibers and the fascicles are distributed in terms of their orientation. This information can then be used to predict what angle of intersection these fibers make. Specifically, each peak in the histogram is identified and the subpopulation is estimated based on the FWHM. These are then assigned to either fibers or fascicles based on observation (i.e., in the xy plane, the image shows that fibers make a vertical/horizontal grid while the fascicles make more of a diagonal/diagonal diversion; this means that the peaks near 0 and 90 (which is equivalent to −90) correspond to the fibers, and the peaks near −45 and 45 correspond to the fascicles). After obtaining the total populations of the entire volume, the intersections are estimated by subtracting the corresponding two peaks and then scaling that result by the FWHM. Then, assuming that all fibers and fascicles have similar pixel counts, the fraction of fibers making certain intersections can be determined.

Autocorrelation Analysis of Myelinated Fibers

The analysis using the finite difference gradient is a nonlinear process that may introduce error to the analysis. The error is compounded by the fact that the z-resolution of the volume is almost three times lower than the x- and y-resolutions. A more accurate approach would be to use autocorrelation. (The finite difference gradient acts as a high pass filter for the autocorrelation.) Autocorrelation would show all the peak distributions in a more non-biased manner. As such, we calculated the autocorrelation in the volume image using MATLAB. Specifically, we used the Fourier convolution theorem with 3DFFT and a periodic boundary condition to calculate the autocorrelation of the volume image filtered with a Gaussian window; then, we transformed the resulting autocorrelation data in Cartesian coordinates to spherical coordinates and integrated out the radial component to visualize the data.

SWITCH-Mediated Antibody Labeling

Samples were first equilibrated in a large volume of SWITCH-Off solution (0.5 mM SDS in PBS). Samples were then moved to a volume a SWITCH-Off solution just large enough to cover the sample and containing 20 μL of antibody solution (for histone H3 staining of 1-mm-thick tissue blocks.) Care should be taken to ensure that the final concentration of SDS in the SWITCH-Off solution is appropriate after the addition of antibody solution. The amount of antibody solution necessary will depend on the target identity. The samples were incubated in this antibody solution for 12 hr at 37° C. with gentle shaking. Samples were then transferred to 10 mL of SWITCH-On solution (PBST) and were washed for 12 hr at 37° C. with gentle shaking

Results

### Synchronizing Dialdehyde-Tissue-Gel Formation Enables Scalable Tissue Preservation

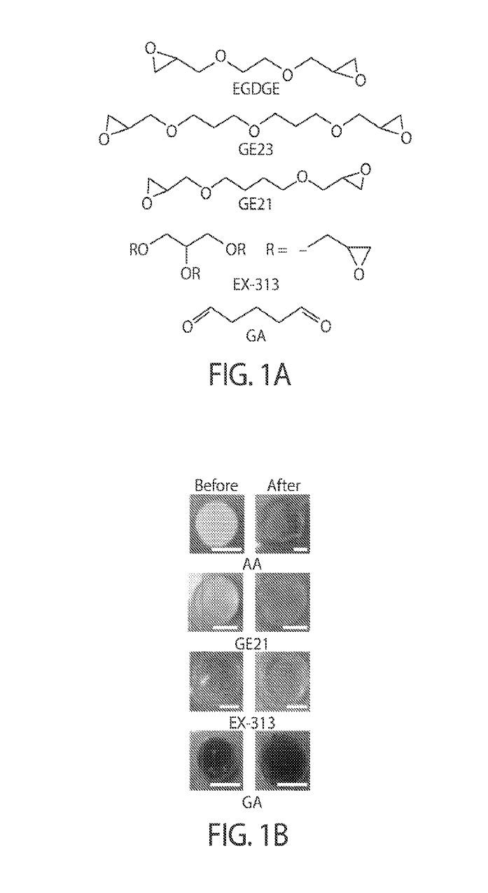

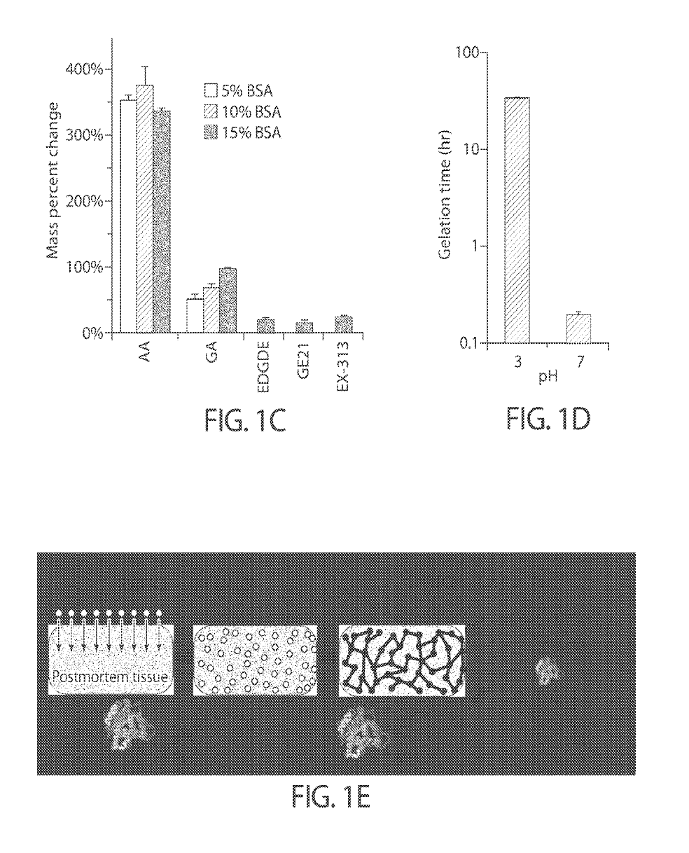

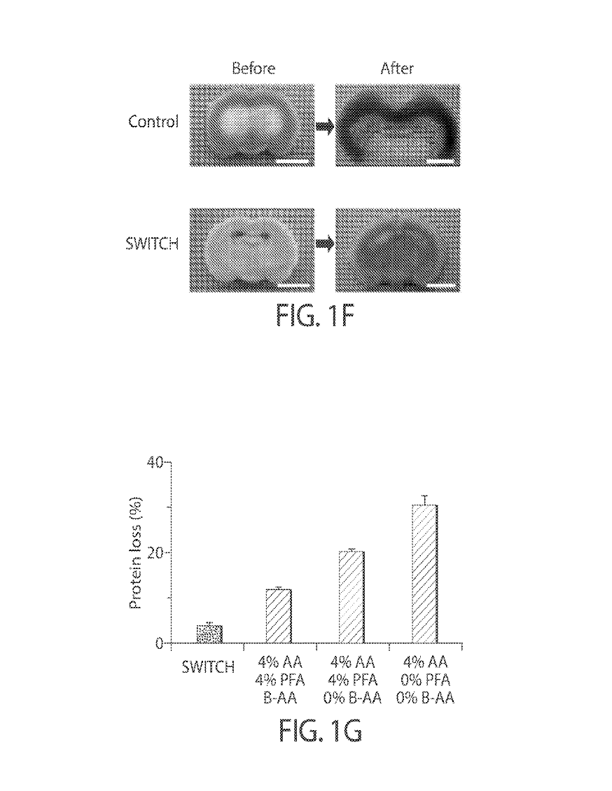

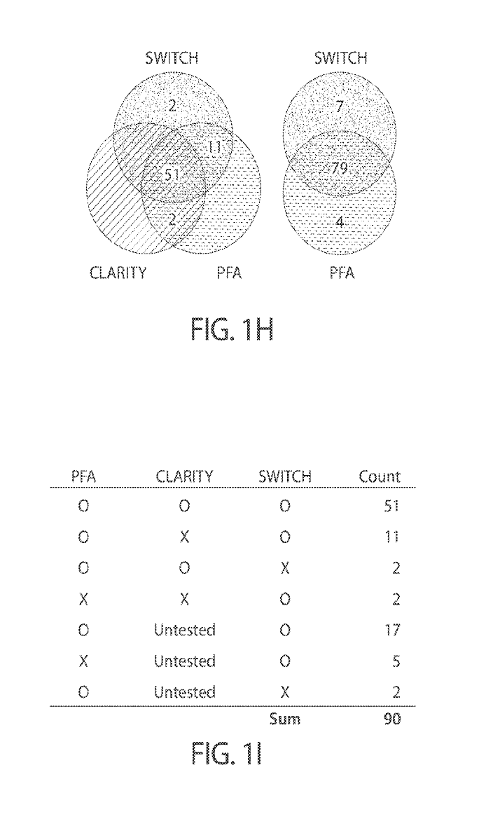

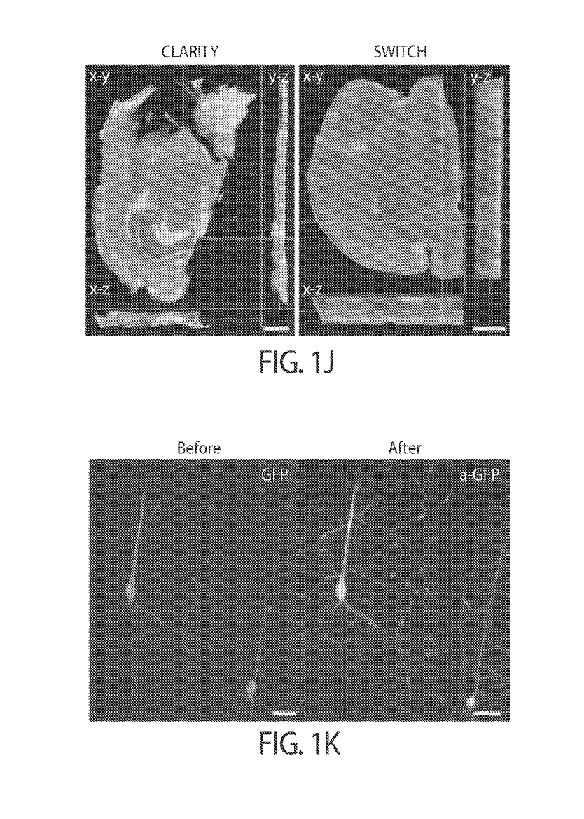

First, we sought to develop a way to transform animal and human samples into a mechanically and chemically stable form for multiplexed imaging. We hypothesized that small, non-ionic, multifunctional crosslinkers might satisfy two key requirements for such a transformation: (1) rapid penetration without the use of perfusion and (2) a high degree of molecular crosslinking to improve sample durability (Hopwood, 1972; Sung et al., 1996). Among many options, we chose to evaluate the following owing to their small size and high water solubility (FIG. 1A): ethylene glycol diglycidyl ether (EGDGE), dipropylene glycol diglycidyl ether (GE23), 1,4-butanediol diglycidyl ether (GE21), glycerol polyglycidyl ether (EX-313), and glutaraldehyde (GA).

We found that all of these chemicals except GE23 formed a solid gel upon incubation with 15% bovine serum albumin (BSA). indicating the formation of a crosslinked network (FIG. 1B). We examined the stability of the gels along with polyacrylamide (AA)-BSA gels by measuring the change in their volume after incubation in a 200 mM SDS solution heated to 80° C. (elution condition). AA-BSA gels swelled and became fragile after exposure to the harsh condition (FIG. 1B and FIG. 1C), whereas multi-functional fixative-BSA gels maintained their structural integrity. In particular, GA-BSA gels showed minimal volume change at a wide range of BSA and GA concentrations, whereas others only gelled at high protein concentrations (FIG. 1C). This result indicates that multifunctional fixatives alone might be sufficient to form a stable matrix that can withstand the harsh elution condition. The average protein content throughout mouse brain samples is around 10% and may be lower within certain regions. Accordingly, we proceeded with the following experiments using GA as the crosslinker.

Next, we asked whether GA can rapidly penetrate tissue to form a uniform tissue-gel without the use of perfusion, which is required for processing most human clinical samples. We incubated a non-fixed whole adult rat brain in PBS containing 1% GA for 2 days and characterized the GA penetration depth and gel formation. Although the small size of GA should make it highly mobile, only the outer layer of the brain was fixed (FIG. 1F). When a coronal slice from the middle of the brain was exposed to the elution condition, the core of the tissue completely disintegrated, indicating that no gel matrix had formed in the center of the brain (FIG. IF). Limited GA penetration has significantly hampered its use in preserving large postmortem tissues (Hopwood, 1967). We suspect that rapid reaction of GA with native biomolecules within the outer layer of the brain may cause depletion of GA molecules before they can reach the core.

To overcome this issue, we sought to control the reaction kinetics of GA and biomolecules throughout the system using the SWITCH approach to achieve uniform tissue preservation.

The GA reaction rate is pH-dependent. When we titrated solutions of GA and BSA to pH 3, GA-BSA gel formation time increased by nearly 200-fold (FIG. 1D). Using this pH dependence, we were able to disperse GA uniformly throughout a sample by switching off the crosslinking reaction with a low-pH buffer (FTG. 1E, left). After 2 days of incubation at low pH, we switched on sample-wide GA-tissue crosslinking by shifting the pH of the sample to a neutral pH (FIG. 1E, right). Using this passive buffer-switching approach, we were able to achieve complete GA penetration and uniform gel formation throughout the entire rat brain (FIG. 1F).

### Dialdehyde-Tissue-Gel Preserves Structural and Molecular Information Effectively

We next asked whether the GA-tissue-gel has mechanical and chemical properties desirable for multiplexing-based proteomic imaging. Proteomic imaging requires (1) high preservation of endogenous biomolecules and their antigenicity, (2) high structural integrity, and (3) minimal tissue damage during repeated cycles of destaining, labeling, and imaging processes.

We first tested whether endogenous biomolecules are well preserved by measuring protein loss after clearing. We found that control tissues lost an average of 30-40% protein and AA-tissue-gel lost 10-20%, but GA-tissue-gel slices lost only 3-5% of their protein content

(FIG. 1G).

We next asked whether antigenicity of the retained biomolecules is well preserved. We tested 90 antibodies, targeting biomolecules of different sizes (single amino acid to proteins) and subcellular localizations (membrane bound, cytoplasm, nucleus, synapses). Surprisingly, 86 of 90 antibodies were compatible with GA-tissue-gel (FIG. 1H and FIG. 1I; Table 1). Note that even small molecules, such as dopamine, which are not typically compatible with PFA-fixation, were observable in GA-tissue-gel after the complete removal of lipid bilayers. These biomolecules were stable against heat and chemical treatment, and their antigenicity was well preserved after exposure to elution conditions.

TABLE 1
Antibody Summary

| | | | | | | | | |
|---|---|---|---|---|---|---|---|---|
| | | | Host | | Target | | | |
| Antigen | Vendor | Catalog # | species | Clonality^{a} | size | PFA^{b} | CLARITY^{b} | SWITCH^{b} |

Cell or nucleus markers

| | | | | | | | | |
|---|---|---|---|---|---|---|---|---|
| Histone H3 | CST | 4499 | Rabbit | M | Protein | ◯ | ◯ | ◯ |
| Histone H3 | CST | 12230 | Rabbit | M | Protein | ◯ | ◯ | ◯ |
| (Alexa 647- | | | | | | | | |
| conjugated) | | | | | | | | |
| Histone H3 | Abcam | ab1791 | Rabbit | P | Protein | ◯ | ◯ | ◯ |
| Histone H3 | Abcam | ab10799 | Mouse | M | Protein | ◯ | Untested | ◯ |
| NeuN | Abcam | ab104225 | Rabbit | P | Protein | ◯ | ◯ | ◯ |
| NeuN | Abcam | ab177487 | Rabbit | M | Protein | ◯ | ◯ | ◯ |
| NeuN | CST | 12943 | Rabbit | M | Protein | ◯ | ◯ | ◯ |
| NeuN | Covance | SIG- | Mouse | M | Protein | ◯ | ◯ | ◯ |
| | | 39860 | | | | | | |

Cell type markers

| | | | | | | | | |
|---|---|---|---|---|---|---|---|---|
| CaMKIIa | Abcam | ab22609 | Mouse | M | Protein | ◯ | Untested | X |
| ChAT | Abcam | ab18736 | Sheep | P | Protein | ◯ | Untested | ◯ |
| GFAP | CST | 3670 | Mouse | M | Protein | ◯ | ◯ | ◯ |
| GFAP | Abcam | ab4674 | Chicken | P | Protein | ◯ | ◯ | ◯ |
| GFAP | Abcam | ab48050 | Rabbit | P | Protein | ◯ | Untested | ◯ |
| GFAP | Abcam | ab68428 | Rabbit | M | Protein | X | Untested | ◯ |
| GFAP | Abcam | ab138519 | Rabbit | P | Protein | ◯ | Untested | ◯ |
| Iba1 | Wako | 019- | Rabbit | P | Protein | ◯ | ◯ | ◯ |
| | | 19741 | | | | | | |
| Iba1 | Abcam | ab5076 | Goat | P | Protein | ◯ | Untested | ◯ |
| Calbindin | CST | 13176 | Rabbit | M | Protein | ◯ | ◯ | ◯ |
| Calbindin | Abcam | ab49899 | Rabbit | P | Protein | X | X | ◯ |
| Calbindin | Abcam | ab11426 | Rabbit | P | Protein | ◯ | ◯ | ◯ |
| Calbindin | Abcam | ab82812 | Mouse | M | Protein | ◯ | Untested | ◯ |
| Calretinin | Abcam | ab702 | Rabbit | P | Protein | ◯ | X | ◯ |
| Calretinin | Abcam | ab92341 | Rabbit | M | Protein | ◯ | X | ◯ |
| Calretinin | Abcam | ab133316 | Rabbit | M | Protein | ◯ | Untested | ◯ |
| EAAT2 | Abcam | ab178401 | Rabbit | M | Protein | ◯ | ◯ | ◯ |
| FOXP2 | Abcam | ab1307 | Goat | P | Protein | ◯ | ◯ | X |
| FOXP2 | Abcam | ab16046 | Rabbit | P | Protein | ◯ | X | ◯ |
| FOXP2 | Abcam | ab58599 | Goat | P | Protein | ◯ | X | ◯ |
| GAD67 | Abcam | ab26116 | Mouse | M | Protein | ◯ | ◯ | ◯ |
| GAD67 | Abcam | ab75712 | Chicken | P | Protein | ◯ | Untested | ◯ |
| GAD67 | Millipore | MAB5406 | Mouse | M | Protein | ◯ | Untested | ◯ |
| Parvalbumin | Abcam | ab32895 | Goat | P | Protein | ◯ | ◯ | ◯ |
| Parvalbumin | Abcam | ab11427 | Rabbit | P | Protein | ◯ | ◯ | ◯ |
| TH | Abcam | ab134461 | Chicken | P | Protein | ◯ | ◯ | ◯ |
| TH | Covance | MMS- | Mouse | M | Protein | ◯ | ◯ | ◯ |
| | | 5210 | | | | | | |
| TH | Abcam | ab75875 | Rabbit | M | Protein | ◯ | ◯ | ◯ |
| TH | Abcam | ab41528 | Rabbit | P | Protein | ◯ | ◯ | ◯ |
| TH | Abcam | ab112 | Rabbit | P | Protein | ◯ | ◯ | ◯ |
| TH | Covance | PRB-515P | Rabbit | P | Protein | ◯ | ◯ | ◯ |

Neurotransmitters and neuromodulators

| | | | | | | | | |
|---|---|---|---|---|---|---|---|---|
| Dopamine | Abcam | ab6427 | Rabbit | P | Small | X | Untested | ◯ |
| | | | | | molecules | | | |
| GABA | Abcam | ab17413 | Guinea | P | Small | X | X | ◯ |
| | | | pig | | molecules | | | |
| Neuropeptide Y | CST | 11976 | Rabbit | M | Peptides | ◯ | ◯ | ◯ |
| Somatostatin | Millipore | MAB354 | Rabbit | M | Protein | ◯ | ◯ | ◯ |
| VIP | Immunostar | #20077 | Rabbit | P | Peptides | ◯ | ◯ | ◯ |

Neural fiber-related markers

| | | | | | | | | |
|---|---|---|---|---|---|---|---|---|
| MAP2 | CST | 8707 | Rabbit | M | Protein | ◯ | ◯ | ◯ |
| MAP2 | Covance | PCK- | Chicken | P | Protein | ◯ | ◯ | ◯ |
| | | 554P | | | | | | |
| Myelin Basic | Abcam | ab7349 | Rat | M | Protein | ◯ | ◯ | ◯ |
| Protein | | | | | | | | |
| Myelin Basic | Abcam | ab24567 | Mouse | M | Protein | ◯ | ◯ | ◯ |
| Protein | | | | | | | | |
| Myelin Basic | Abcam | ab134018 | Chicken | P | Protein | ◯ | ◯ | ◯ |
| Protein | | | | | | | | |
| Myelin Basic | Aves Labs | MBP | Chicken | P | Protein | ◯ | ◯ | ◯ |
| Protein | | | | | | | | |
| Neurofilament | Abcam | ab92539 | Rabbit | M | Protein | ◯ | ◯ | X |
| 160 kD | | | | | | | | |
| Neurofilament | Abcam | ab7794 | Mouse | M | Protein | ◯ | ◯ | ◯ |
| 160 kD | | | | | | | | |
| Neurofilament | Abcam | ab64300 | Rabbit | P | Protein | ◯ | ◯ | ◯ |
| 160 kD | | | | | | | | |
| Neurofilament | Abcam | ab4680 | Chicken | P | Protein | ◯ | ◯ | ◯ |
| 200 kD | | | | | | | | |
| Neurofilament | Abcam | ab8135 | Rabbit | P | Protein | ◯ | ◯ | ◯ |
| 200 kD | | | | | | | | |
| Neurofilament L | CST | 2837 | Rabbit | M | Protein | ◯ | ◯ | ◯ |
| Neurofilament | Covance | SMI-312P | Mouse | P | Protein | ◯ | ◯ | ◯ |
| SMI-312P | | | | | | | | |
| Neurofilament | Covance | SMI-312R | Mouse | P | Protein | ◯ | ◯ | ◯ |
| SMI-312R | | | | | | | | |
| Neurofilament | Covance | SMI-32P | Mouse | P | Protein | ◯ | X | ◯ |
| SMI-32P | | | | | | | | |
| Neurofilament | Covance | SMI-32R | Mouse | P | Protein | ◯ | X | ◯ |
| SMI-32R | | | | | | | | |
| Neuronal | Covance | PRB-435P | Rabbit | P | Protein | ◯ | ◯ | ◯ |
| Class III β- | | | | | | | | |
| tubulin | | | | | | | | |
| Neuronal | Covance | A488- | Mouse | P | Protein | ◯ | X | ◯ |
| Class III β- | | 435L | | | | | | |
| tubulin (Alexa | | | | | | | | |
| 488- | | | | | | | | |
| conjugated) | | | | | | | | |
| Neurofilament H | CST | 2836BF | Mouse | M | Protein | ◯ | ◯ | ◯ |

Neurogenesis markers

| | | | | | | | | |
|---|---|---|---|---|---|---|---|---|
| ER81 | Covance | PRB- | Rabbit | P | Protein | ◯ | ◯ | ◯ |
| | | 362C | | | | | | |
| Ki67 | Abcam | ab16667 | Rabbit | M | Protein | ◯ | ◯ | ◯ |

Receptors

| | | | | | | | | |
|---|---|---|---|---|---|---|---|---|
| Estrogen | Abcam | ab32063 | Rabbit | M | Protein | ◯ | X | ◯ |
| receptor alpha | | | | | | | | |
| Glutamate | Abcam | ab86141 | Rabbit | P | Protein | ◯ | Untested | ◯ |
| Receptor 1 | | | | | | | | |
| Glutamate | Abcam | ab109450 | Rabbit | M | Protein | X | Untested | ◯ |
| Receptor 1 | | | | | | | | |
| NMDAR1 | Abcam | ab109182 | Rabbit | M | Protein | X | Untested | ◯ |

Exogenous proteins

| | | | | | | | | |
|---|---|---|---|---|---|---|---|---|
| GFP | Life | A11122 | Rabbit | P | Protein | ◯ | ◯ | ◯ |
| | Technologies | | | | | | | |
| GFP (Alexa | Life | A21311 | Rabbit | P | Protein | ◯ | ◯ | ◯ |
| 488 | Technologies | | | | | | | |
| conjugated) | | | | | | | | |
| GFP (Alexa | Life | A31851 | Rabbit | P | Protein | ◯ | ◯ | ◯ |
| 555 | Technologies | | | | | | | |
| conjugated) | | | | | | | | |
| GFP (Alexa | Life | A21312 | Rabbit | P | Protein | ◯ | ◯ | ◯ |
| 594 | Technologies | | | | | | | |
| conjugated) | | | | | | | | |
| GFP (Alexa | Life | A31852 | Rabbit | P | Protein | ◯ | ◯ | ◯ |
| 647 | Technologies | | | | | | | |
| conjugated) | | | | | | | | |
| RFP | Rockland | 600-401- | Rabbit | P | Protein | ◯ | Untested | ◯ |
| | | 379 | | | | | | |

Synaptic proteins

| | | | | | | | | |
|---|---|---|---|---|---|---|---|---|
| PSD-95 | Abcam | ab12093 | Goat | P | Protein | ◯ | ◯ | ◯ |
| Synapsin I | CST | 5297 | Rabbit | M | Protein | ◯ | ◯ | ◯ |
| Synaptophysin | Abcam | ab8049 | Mouse | M | Protein | ◯ | ◯ | ◯ |
| Synapsin I | Abcam | ab64581 | Rabbit | P | Protein | ◯ | Untested | ◯ |
| Synaptophysin | CST | 5461 | Rabbit | M | Protein | ◯ | Untested | ◯ |
| Synaptophysin | Abcam | ab32127 | Rabbit | M | Protein | ◯ | Untested | ◯ |
| Synaptophysin | Abcam | ab52636 | Rabbit | M | Protein | ◯ | Untested | ◯ |
| SYNPR | Abcam | ab175224 | Rabbit | M | Protein | ◯ | Untested | ◯ |

Disease-related markers

| | | | | | | | | |
|---|---|---|---|---|---|---|---|---|
| β-Amyloid | CST | 2454 | Rabbit | P | Protein | ◯ | ◯ | ◯ |

Others

| | | | | | | | | |
|---|---|---|---|---|---|---|---|---|
| FOXP3 | Abcam | ab20034 | Mouse | M | Protein | ◯ | Untested | X |
| PGP9.5 | Abcam | ab10404 | Rabbit | P | Protein | ◯ | X | ◯ |
| PGP9.5 | Abcam | ab108986 | Rabbit | M | Protein | ◯ | X | ◯ |
| PGP9.5 | Abcam | ab109261 | Rabbit | M | Protein | ◯ | X | ◯ |
| Reelin | Abcam | ab78540 | Mouse | M | Protein | ◯ | Untested | ◯ |
| Reelin | Abcam | ab138370 | Rabbit | P | Protein | X | Untested | ◯ |

^{a}M, monoclonal; P, polyclonal.
^{b}A working dilution of 1:100 was used for all antibody testing.

Good structural preservation is essential for resolving protein location with high precision and for studying molecular interrelationships. To characterize the macroscale structural preservation of the samples, we cleared 1-mm-thick tissue blocks using the elution condition and visualized their structural deformation (FIG. 1J). The PFA-only tissue completely disintegrated. Even the AA-tissue-gel exhibited large deformations overall. GA-tissue-gel, however, showed no signs of structural damage throughout the entirety of the sample.

We next examined structural preservation on a microscopic scale. We imaged green fluorescent protein (GFP)-expressing neurons in the cortex of a PFA-fixed 1-mm-thick thy1-EGFP M line block (FIG. 1K). We then SWITCH-processed the tissue, cleared it using the harsh elution condition, stained it against GFP, and imaged the same neurons. As shown in FIG. 1K, the microscopic morphology of the neurons was well preserved throughout the entire process. These results show GA-tissue-gel may be ideal for highly multiplexed structural and molecular phenotyping.

### SWITCH and Robust Computational Algorithms Enable Highly Multiplexed Imaging at Single-Cell Resolution

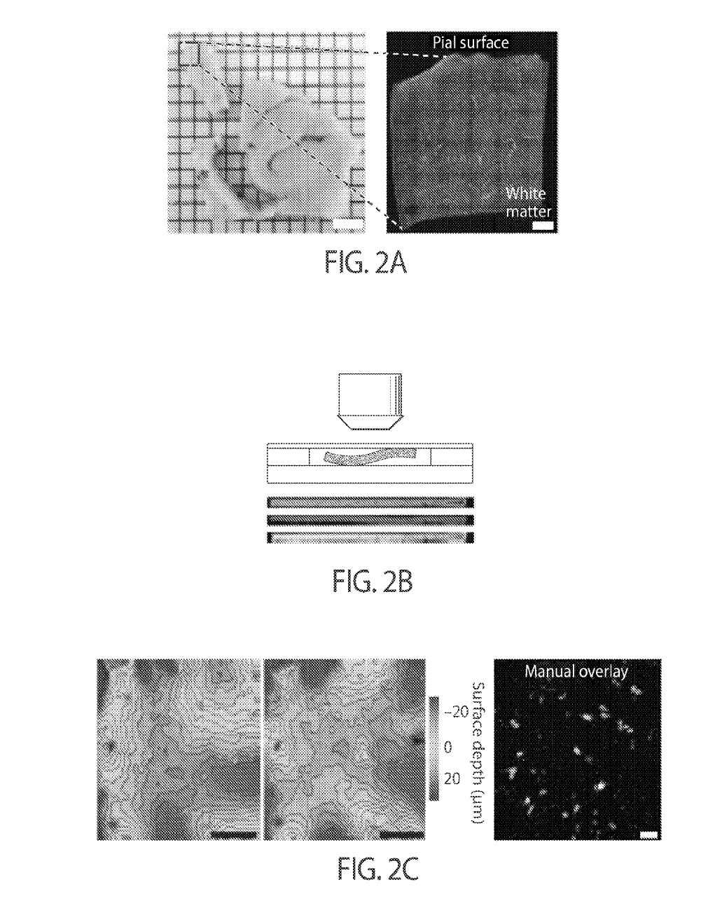

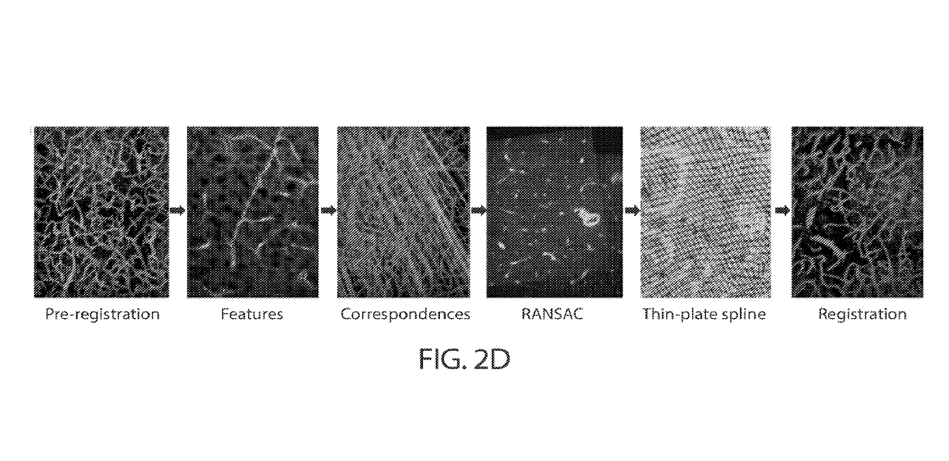

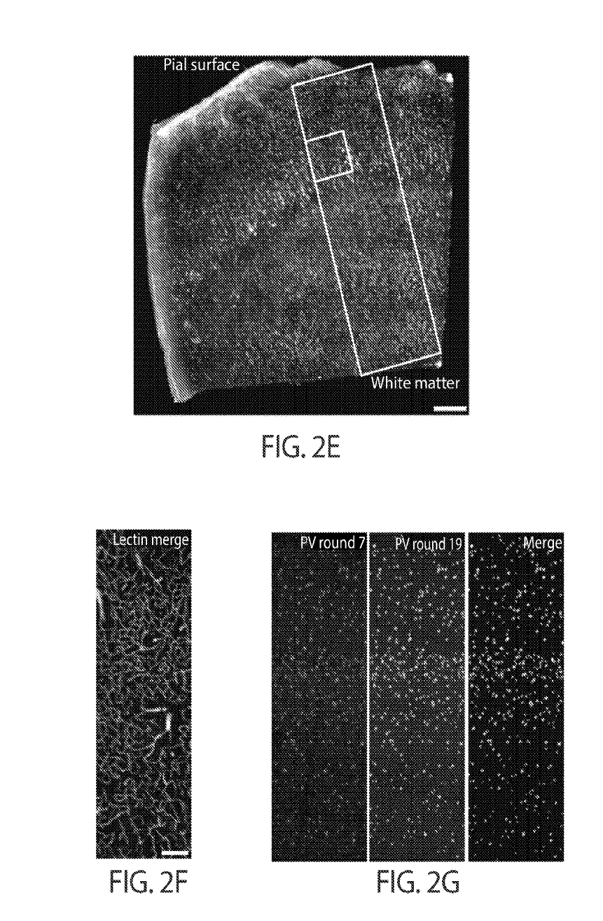

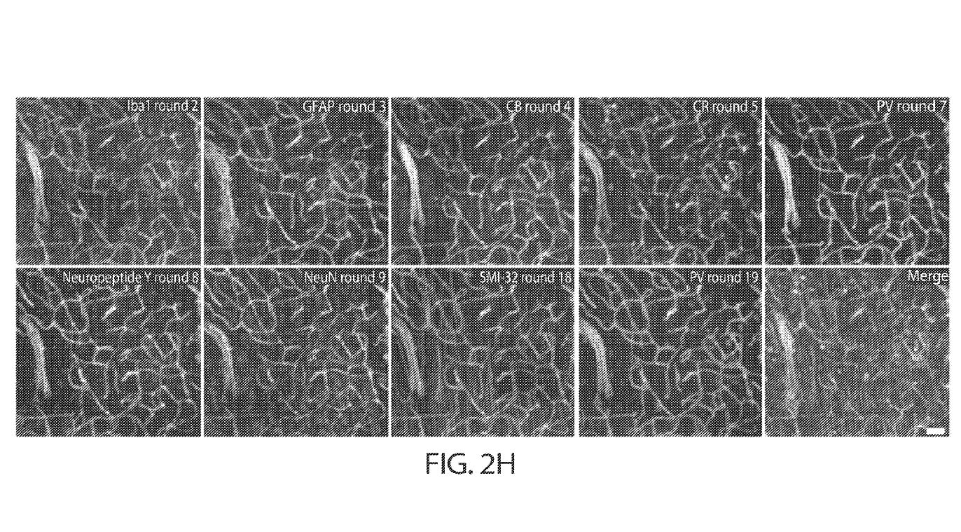

Interrogating the three-dimensional (3D) distribution of molecules, cells, and the overall tissue organization requires precise co-registration of multiple volume images. We first asked if simple manual overlay of two datasets allows precise co-registration. As a stringent test, we used datasets from multi-round imaging of a SWITCH-processed 100-μm-thick human brain slice (100 μm×3,200 μm×3,200 μm) (FIG. 2A). The high aspect ratio of such tissues makes it more prone to physical warping, which renders co-registration particularly challenging. We first stained the tissue using DAPI and anti-parvalbumin (PV) antibody. The slice was then enclosed in a space larger than the tissue to exaggerate possible tissue deformation in the mounting process (FIG. 2B). After imaging, the sample was exposed to the elution condition overnight (O/N) to completely remove imaged probes. We then restained the tissue using the same probes and repeated the imaging process. Note that only GA-tissue-gels could maintain their integrity against the elution treatment. Both AA-tissue-gels and PFA-fixed samples deteriorated rapidly in the same condition.

As predicted, a large degree of tissue warping in the mounting process (FIG. 2C) made manual overlay insufficient for the task of interrogating a tissue across multiple staining rounds. To achieve precise co-registration of volume images in the presence of such high-degree warping, we custom-designed a robust computational software based on a feature-detection approach that was ideal for our experimental procedure (FIG. 2D). Each staining round contained one fluorescence channel devoted to a lectin stain because the morphology of blood vessels creates distinctive keypoints that computer vision algorithms are well suited to identify. With the keypoints, the algorithm warps the tissue in a physically plausible manner into the correct position.

As a stringent test of the algorithm, we used the same SWITCH-processed human sample with the high aspect ratio (FIG. 2A). For each round, the sample was stained with DAPI, lectin, and one antibody to label a target protein. Although at least three antibodies can be used for each round in addition to lectin and DAPI , we chose to use one antibody for each round to eliminate any possible cross-talk between channels. After acquiring images, we destained the sample and began the next round of labeling. We repeated the above procedure 22 times using markers for various cell types (FIG. 2H; Table 2). Staining was not successful in every round due to the use of non-validated antibodies, sub-optimal staining conditions, or human error, all of which often occur in general laboratory settings and can result in the loss of important samples. However, a SWITCH-processed sample is free from this issue as the tissue can be washed and reused repeatedly.

TABLE 2
Multiplexed Imaging Rounds

| | Excitation wavelength (nm) | | |
|---|---|---|---|
| Round | 405 | 488 | 647^{a} |
| 1 | DAPI | Lectin | Iba1 |
| 2 | DAPI | Lectin | Iba1 |
| 3 | DAPI | Lectin | GFAP |
| 4 | DAPI | Lectin | Calbindin |
| 5 | DAPI | Lectin | Calretinin |
| 6 | DAPI | Lectin | Fluoromyelin |
| 7 | DAPI | Lectin | Parvalbumin |
| 8 | DAPI | Lectin | Neuropeptide Y |
| 9 | DAPI | Lectin | NeuN |
| 10 | DAPI | Lectin | DiI D7777 |
| 11 | DAPI | Lectin | SOM |
| 12 | DAPI | Lectin | Cholecystokinin |
| 13 | DAPI | Lectin | NMDAR1 |
| 14 | DAPI | Lectin | NC3βT |
| 15 | DAPI | Lectin | GAD67 |
| 16 | DAPI | Lectin | Npas4 |
| 17 | DAPI | Lectin | NAChR |
| 18 | DAPI | Lectin | SMI-32P |
| 19 | DAPI | Lectin | Parvalbumin |
| 20 | DAPI | Lectin | Iba1 |
| 21 | DAPI | Lectin | GFAP |
| 22 | DAPI | Lectin | SMI-312 |

^{a}iba1, ionized calcium-binding adapter molecule 1; GFAP, glial fibrillary acidic protein; SOM, somatostatin; NMDAR1, N-methyl-D-aspartate receptor 1; NC3βT, neuronal class III β-tubulin; GAD67, glutamic acid decarboxylase 67; Npas4, neuronal PAS domain protein 4; NAChR, nicotinic acetylcholine receptor.

We were able to successfully co-register all 9 datasets with successful staining (FIG. 2E and FIG. 2H). We asked whether changes in the sample might be occurring between staining rounds. To test this, we repeated staining with anti-PV antibodies in rounds 7 and 19 and co-registered the resulting datasets. Even when separated by 12 rounds of labeling, we were able to achieve single-cell accuracy of registration with 99% agreement between the two rounds (FIG. 2G).

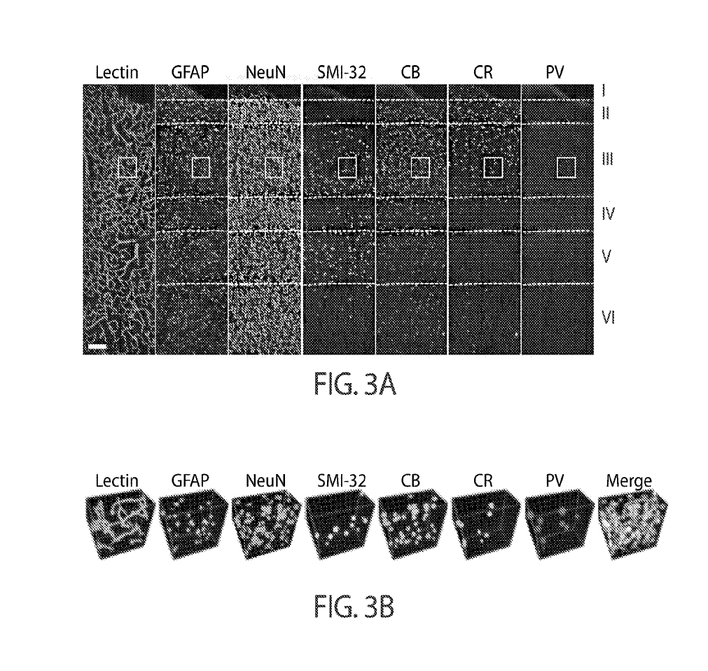

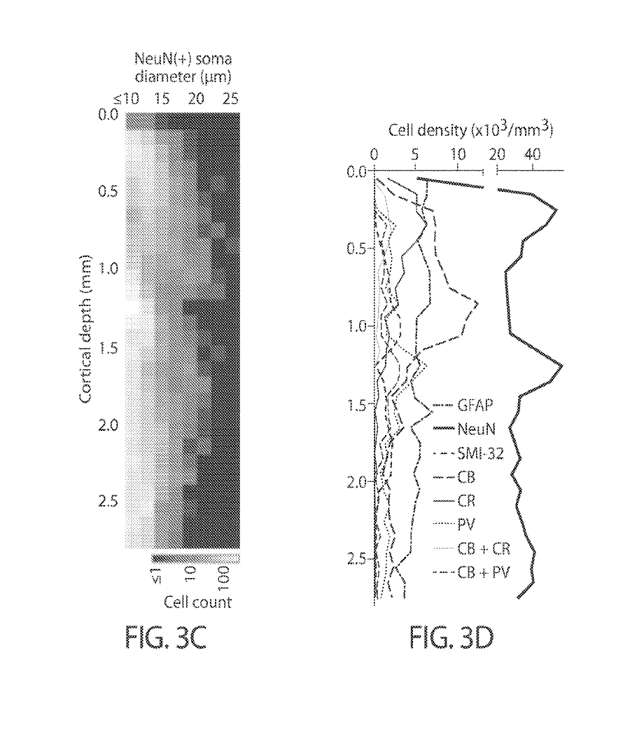

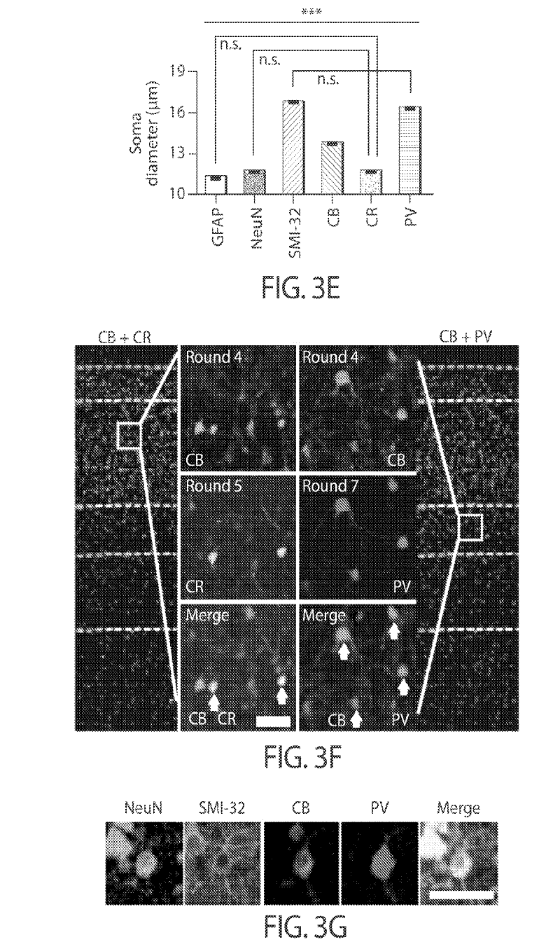

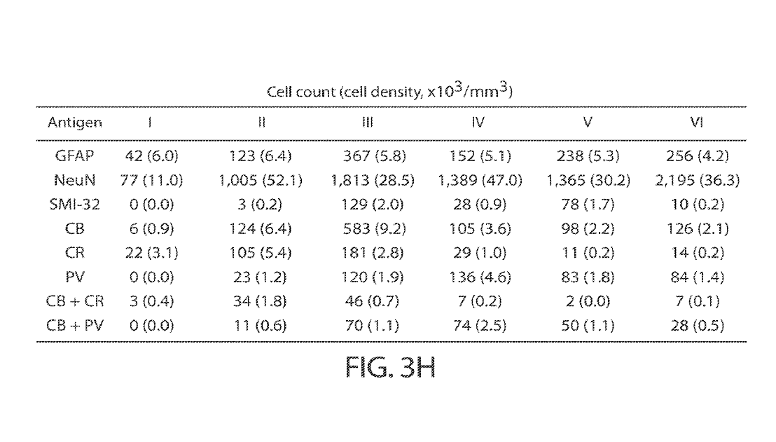

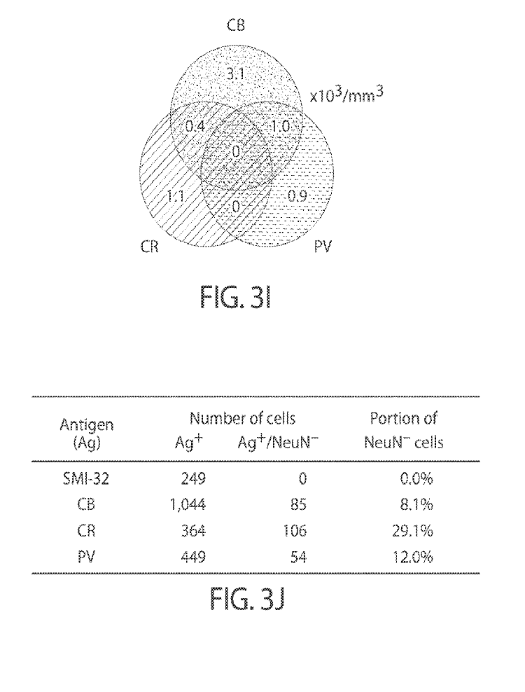

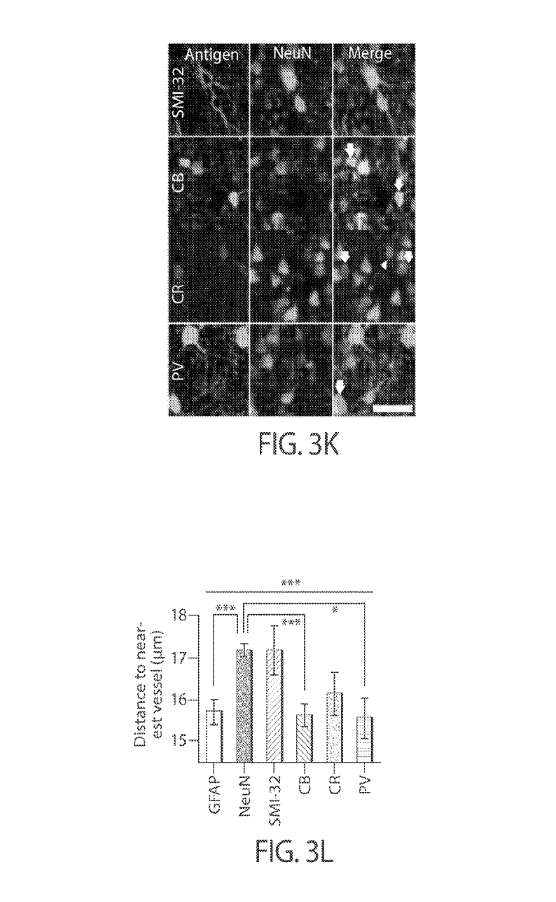

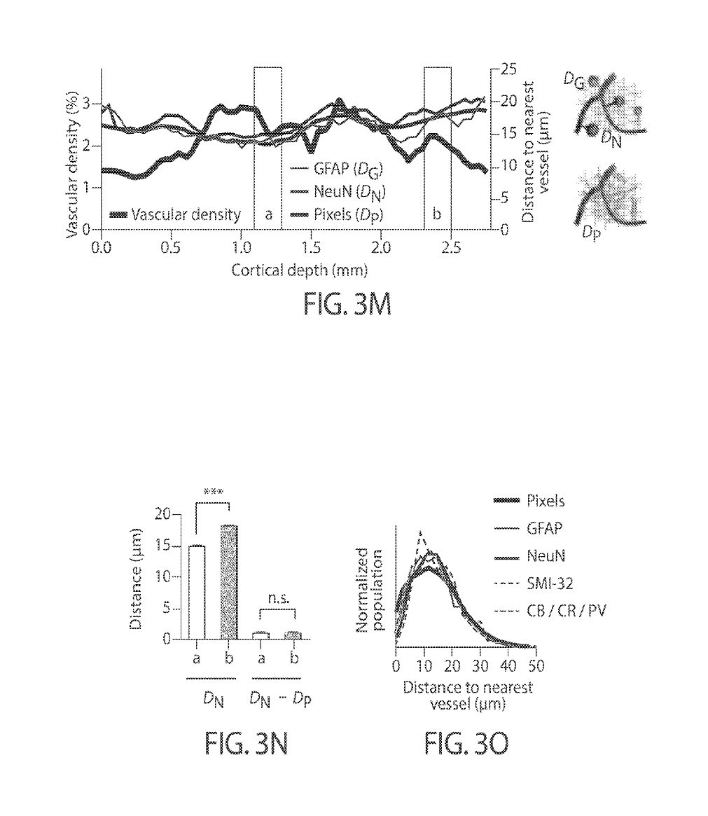

We next performed joint statistical analysis of the integrated cross-talk—free dataset to extract diverse phenotypic information from human brain (FIGS. 3A-3)). We included lectin, GFAP, NeuN, SMI-32, and three calcium-binding protein channels—calbindin (CB), calretinin (CR), and PV—in the quantitative analysis. First, we used semi-automated algorithms to identify blood vessels and cells expressing the target antigens (FIG. 3A and FIG. 3B) and extract their spatial (x, y, z coordinates) and morphological (e.g., cell soma size) information. Density and size profiles of NeuN-positive cells (FIG. 3C and FIG. 3D) enabled us to define the cortical layers (FIG. 3A) according to established criteria (De Sousa et al., 2010). NeuN⁺ density was high in cortical layers II and IV, with characteristic small cells (NeuN in FIGS. 3A, 3C, 3D, and 3H). Large NeuN⁺ neurons were concentrated in layers III and V. A portion of these were large pyramidal neurons positive for SMI-32 (FIG. 3A, FIG. 3E, and FIG. 3H). CB⁺, CR⁺, and PV⁺ cells also showed distinct distribution patterns along the cortical axis (FIG. 3A and FIG. 3D), in agreement with previous studies (DeFelipe et al., 1999; Leuba et al., 1998).

We next performed unbiased combinatorial expression profiling with the 6 cell-type specific proteins (GFAP, NeuN, SMI-32, CB, CR, PV). Among 63 possible combinations, 16 were found (Table 3). We identified sub-populations of CB⁺/CR⁺ and CB⁺/PV⁺ cells, but no CR⁺/PV⁺ or CB⁺/CR⁺/PV⁺ cells (FIG. 3F, FIG. 3H, and FIG. 3I), in agreement with a previous report regarding mouse visual cortex (Gonchar et al., 2007). Interestingly, we observed that a significant portion of the CB, CR, and PV-positive neurons do not express detectable levels of NeuN, a widely used pan-neuronal marker (FIG. 3J and FIG. 3K) (Mullen et al., 1992). In particular, a majority of CR⁺ cells showed very weak (FIG. 3K, arrowhead) or no NeuN immunoreactivity (29.1%), whereas all SMI-32⁺ cells (FIG. 3J and FIG. 3K) were NeuN-positive. These results suggest that NeuN expression may he neuronal-type-specific in adult human visual association cortex. We also found a small number of CB⁺ cells and PV⁺ cells co-expressing SMI-32, a widely used pyramidal neuronal marker (Table 3) (Campbell and Morrison, 1989). Five CB⁺/PV⁺ cells were identified as quadruple-positive (NeuN⁺/SMI-32⁺/CB⁺/PV⁺) cells (FIG. 3G). All of the CB⁺ cells and Pr cells co-expressing SMI-32 were localized in cortical layers III and IV. These results demonstrate the power of SWITCH as a tool for 3D proteomic profiling of intact biological samples at single cell resolution.

Structural relationships between vasculature and brain cells have been a topic of interest in a broad range of basic and clinical research. Many previous studies obtained the cell-to-vessel distance from 2D images or small tissue volumes, which may hinder precise measurement of such 3D properties. Moreover, in many studies, separate measurements from different tissues needed to be compared without considering individual variabilities in local vasculature geometry. There has been no direct comparison of 3D cell-to-vessel distance among diverse cell types within the same intact tissue.

Using the proteomic imaging capability of SWITCH, for the first time, we were able to directly measure cell-to-vessel distances for six different cell types within a single intact tissue (FIG. 3L-FIG. 3O). As expected (McCaslin et al., 2011), GFAP⁺ astrocytes had a shorter mean distance than NeuN⁺ neurons (FIG. 3L). CB⁺ and Pr cells were also more closely localized near blood vessels than NeuN⁺ cells, but the difference was relatively small. FIG. 3M shows that vascular density is not uniform along the cortex. However, the extravascular pixel-to-vessel distance (Dₚ), which we defined as a reference parameter to reflect the effect of the 3D vascular geometry (FIG. 3M, right panel), did not show an inverse relationship with vascular density. This result may suggest that 3D vessel geometry is an important parameter to be considered in understanding a given vascular environment. In fact, cell-to-vessel distance profiles of many cell types closely followed the Dₚ profile (GFAP⁺, D_{G}, and NeuN⁺, D_{N}, shown in FIG. 3M). In particular, when Dₚ was subtracted from cell-to-vessel distances (Dₓ) to cancel the influence of vascular geometric variation, Dₓ-Dₚ turns out to be very consistent throughout cortical depth (FIG. 3N). We further examined the distance distribution profiles for all cell types (FIG. 3O). All profiles showed similar characteristic curves, which can be seen when objects are randomly located in a 3D space (Manzo et al., 2014). We could not observe any cell-type-specific distribution profile or hi- or multi-modal distribution pattern in this sample. Together, these data demonstrate that SWITCH can be used for high-dimensional quantitative phenotyping of human clinical samples.

TABLE 3
Statistics of All Combinations of Six Cell Markers

| Antigen | | | | | | Cortical layer | | | | | |
|---|---|---|---|---|---|---|---|---|---|---|---|
| GFAP | NeuN | SMI-32 | CB | CR | PV | I | II | III | IV | V | VI |
| − | − | − | − | − | + | 0 | 3 | 11 | 5 | 1 | 7 |
| − | − | − | − | + | − | 4 | 15 | 42 | 7 | 5 | 1 |
| − | − | − | − | + | + | None | | | | | |
| − | − | − | + | − | − | 2 | 0 | 14 | 1 | 3 | 8 |
| − | − | − | + | − | + | 0 | 2 | 4 | 5 | 1 | 15 |
| − | − | − | + | + | − | 1 | 11 | 12 | 3 | 1 | 3 |
| − | − | − | + | + | + | None | | | | | |
| − | − | + | − | − | − | None | | | | | |
| − | − | + | − | − | + | None | | | | | |
| − | − | + | − | + | − | None | | | | | |
| − | − | + | − | + | + | None | | | | | |
| − | − | + | + | − | − | None | | | | | |
| − | − | + | + | − | + | None | | | | | |
| − | − | + | + | + | − | None | | | | | |
| − | − | + | + | + | + | None | | | | | |
| − | + | − | − | − | − | 59 | 828 | 1,007 | 1,197 | 1,158 | 2,030 |
| − | + | − | − | − | + | 0 | 8 | 38 | 57 | 32 | 49 |
| − | + | − | − | + | − | 15 | 56 | 93 | 15 | 4 | 6 |
| − | + | − | − | + | + | None | | | | | |
| − | + | − | + | − | − | 1 | 77 | 448 | 22 | 43 | 83 |
| − | + | − | + | − | + | 0 | 10 | 64 | 66 | 49 | 13 |
| − | + | − | + | + | − | 2 | 23 | 34 | 4 | 1 | 4 |
| − | + | − | + | + | + | None | | | | | |
| − | + | + | − | − | − | 0 | 3 | 122 | 24 | 78 | 10 |
| − | + | + | − | − | + | 0 | 0 | 1 | 0 | 0 | 0 |
| − | + | + | − | + | − | None | | | | | |
| − | + | + | − | + | + | None | | | | | |
| − | + | + | + | − | − | 0 | 0 | 4 | 1 | 0 | 0 |
| − | + | + | + | − | + | 0 | 0 | 2 | 3 | 0 | 0 |
| − | + | + | + | + | − | None | | | | | |
| − | + | + | + | + | + | None | | | | | |
| + | − | − | − | − | − | 42 | 123 | 367 | 152 | 238 | 256 |

| | | | | | | |
|---|---|---|---|---|---|---|
| + | − | − | − | − | + | None |
| + | − | − | − | + | − | None |
| + | − | − | − | + | + | None |
| + | − | − | + | − | − | None |
| + | − | − | + | − | + | None |
| + | − | − | + | + | − | None |
| + | − | − | + | + | + | None |
| + | − | + | − | − | − | None |
| + | − | + | − | − | + | None |
| + | − | + | − | + | − | None |
| + | − | + | − | + | + | None |
| + | − | + | + | − | − | None |
| + | − | + | + | − | + | None |
| + | − | + | + | + | − | None |
| + | − | + | + | + | + | None |
| + | + | − | − | − | − | None |
| + | + | − | − | − | + | None |
| + | + | − | − | + | − | None |
| + | + | − | − | + | + | None |
| + | + | − | + | − | − | None |
| + | + | − | + | − | + | None |
| + | + | − | + | + | − | None |
| + | + | − | + | + | + | None |
| + | + | + | − | − | − | None |
| + | + | + | − | − | + | None |
| + | + | + | − | + | − | None |
| + | + | + | − | + | + | None |
| + | + | + | + | − | − | None |
| + | + | + | + | − | + | None |
| + | + | + | + | + | − | None |
| + | + | + | + | + | + | None |

### SWITCH Enables Simple, Rapid, and Scalable Tissue-Clearing

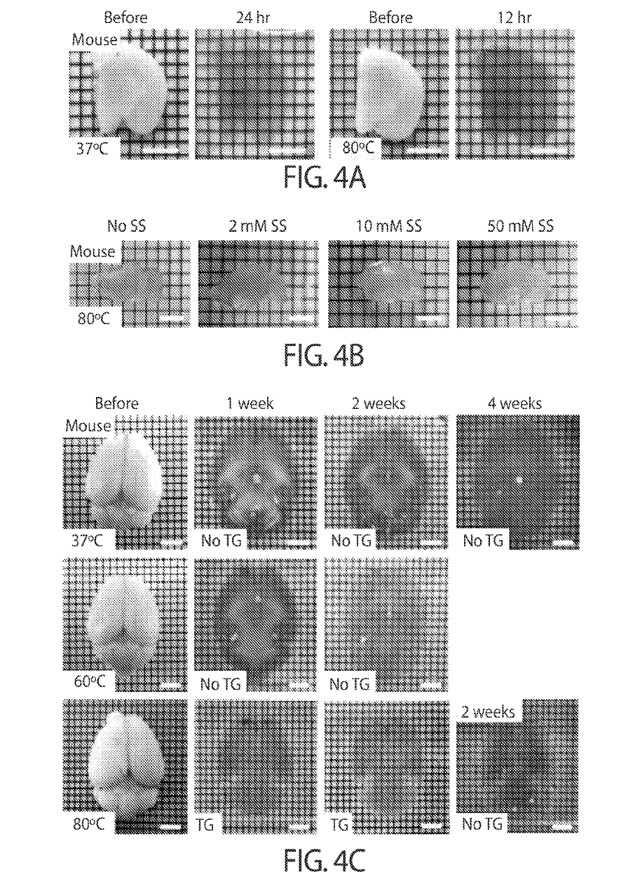

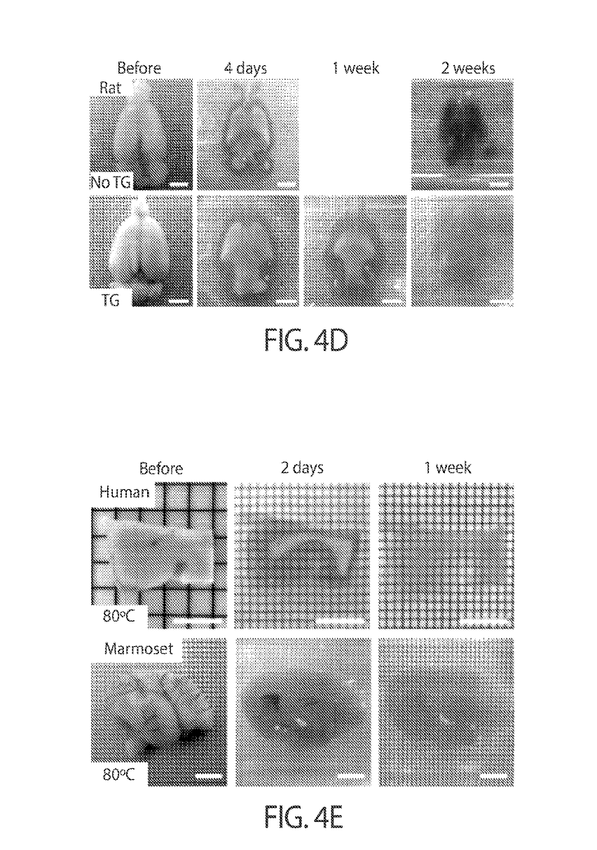

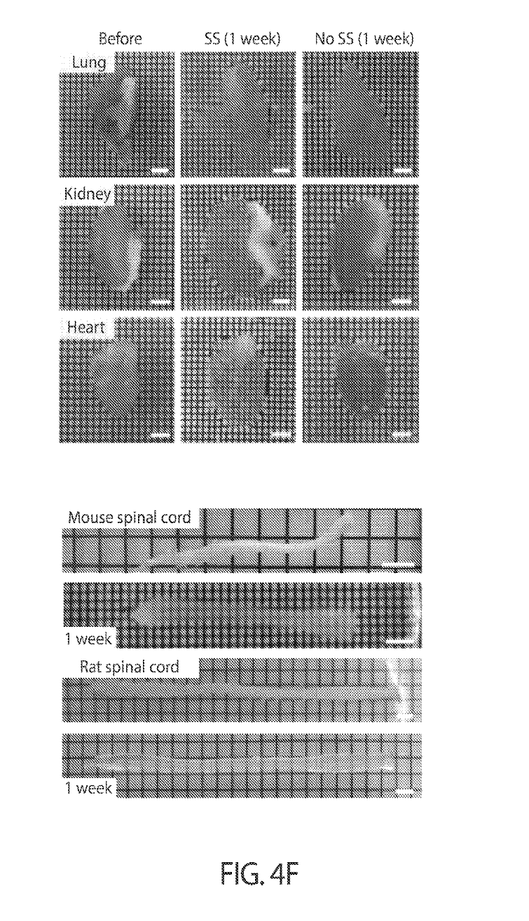

To extend the multiplexed imaging capability of the SWITCH method to large systems, we developed a simple and rapid clearing method. We hypothesized that key steps in detergent-mediated lipid removal, such as permeation of SDS through membranes, might be strongly enhanced by increasing temperature (Keller et al., 2006), and SWITCH-processed samples may endure prolonged incubation at elevated temperatures. Indeed, thermal energy drastically increased the passive clearing speed of SWITCH-processed samples without noticeable tissue damage (FIG. 4A). We achieved passive clearing of a whole adult mouse brain within 4 days at 80° C. (vs. 4 weeks at 37° C.) (FIG. 4C).

Upon prolonged exposure to high temperatures, however, samples developed a brownish hue (Friedman, 1996), which may interfere with imaging at certain wavelengths (FIG. 4B-FIG. 4D and FIG. 4F). We found that reducing agents, such as sodium sulfite and 1-thioglycerol, effectively mitigate tissue browning during thermal clearing (FIG. 4B-FIG. 4D). Using thermal clearing with the reducing agents, we successfully cleared intact adult rat brains (2 weeks) as well as human (1 week) and marmoset samples (1 week), demonstrating the versatility and scalability of the method (FIG. 4D and FIG. 4E). Clearing of various rodent organs was also demonstrated with lung, kidney, heart, liver, and spinal cord (FIG. 4F). The efficacy of sodium sulfite as an anti-browning agent was seen across all tissues.

### SWITCH Enables Visualization and Quantitative Analysis of Entire Myelinated Fiber Tracts

We also sought to apply SWITCH to characterizing myelinated fiber pathways in the brain. Visualizing and analyzing neural fibers with high-resolution light microscopy can provide valuable insights into many studies (Thomas et al., 2014; Wedeen et al., 2012; Zuccaro and Arlotta, 2013), such as validating diffusion tensor imaging (DTI) and understanding the organizing principles of brain connectivity. Furthermore, quantitative analysis of myelinated fibers in 3D may benefit clinical studies and development of novel treatments for many demyelinating diseases (Steinman, 1999), such as multiple sclerosis and transverse myelitis. However, current methods for myelinated fiber visualization require either genetic labeling or a large amount of costly antibodies, limiting their utility to animal tissues or small clinical samples (Wedeen et al., 2012).

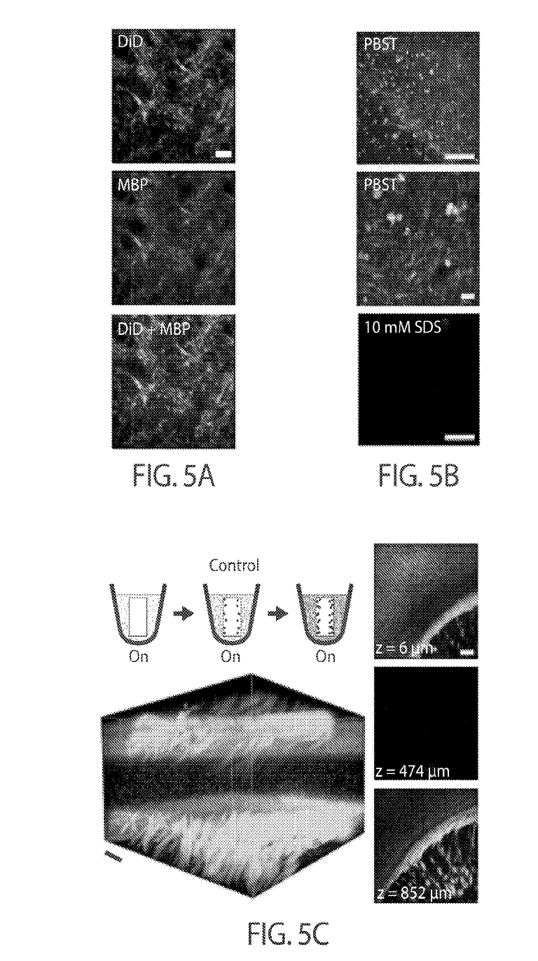

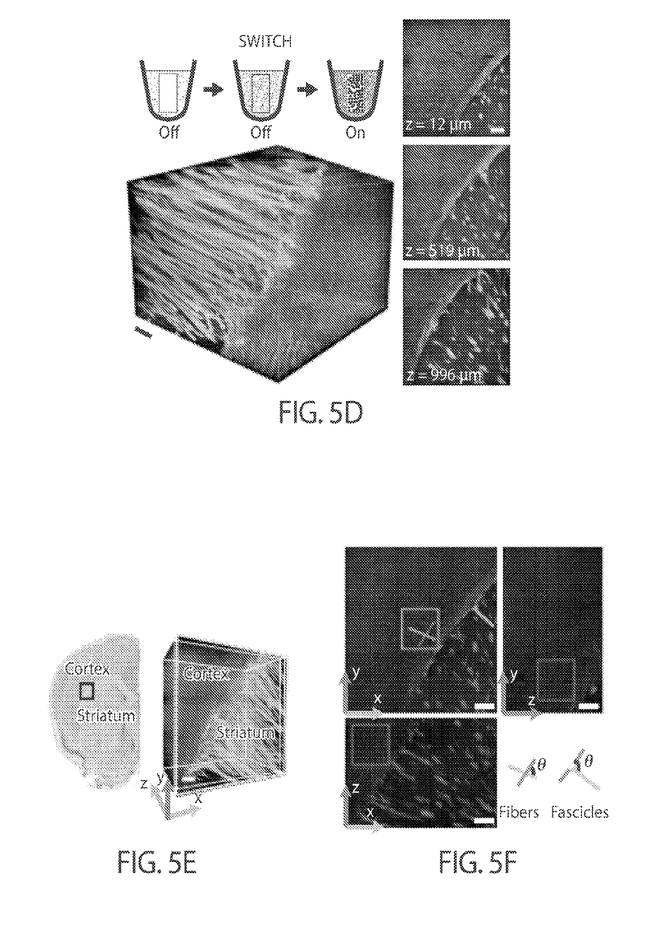

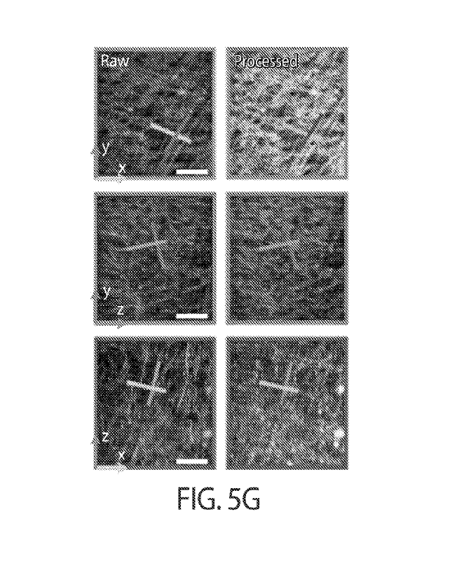

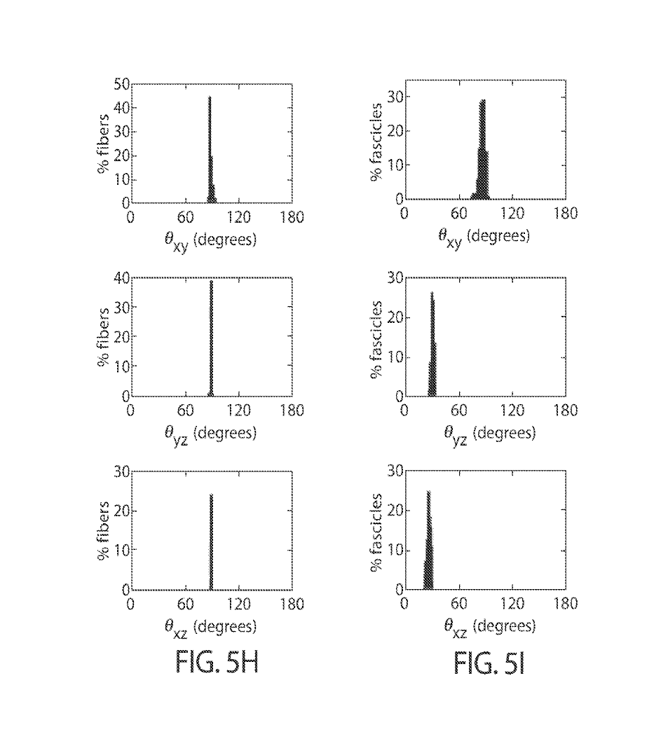

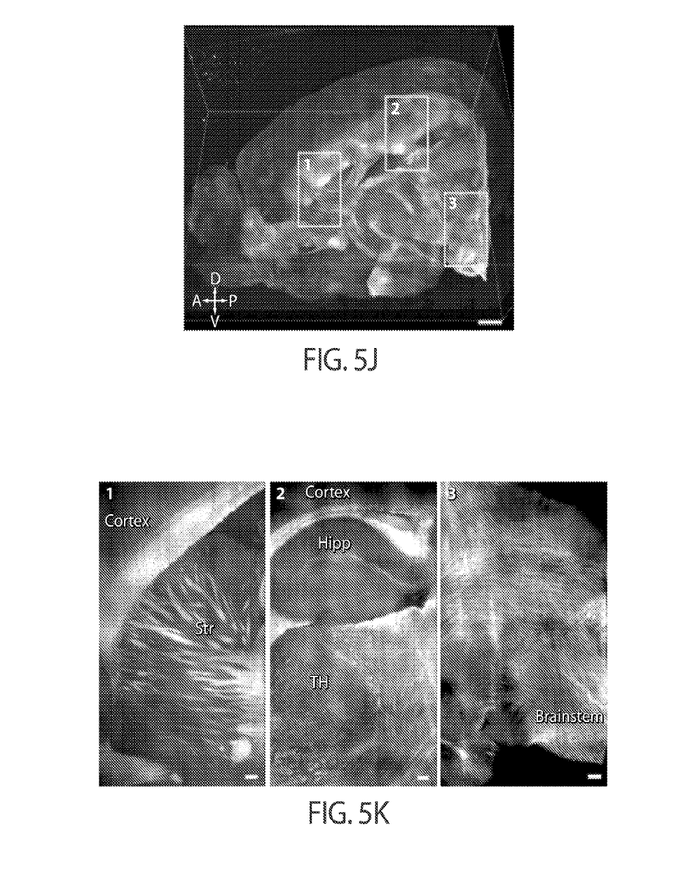

We discovered that a subset of lipids preserved in SWITCH-processed tissues (Hopwood, 1972; Roozemond, 1969) allows lipophilic dyes to selectively visualize lipid-rich membranes (Schlessinger et al., 1977). In particular, we found that long-chain dialkylcarbocyanines robustly stain myelinated axons (FIG. 5A). However, when we attempted to label an intact tissue using conventional methods, we could not achieve dye penetration deeper than 100 μm because dye molecules were depleted as they rapidly associated with abundant targets in the outer layer (FIG. 5C).

We hypothesized that SWITCH may enable rapid and uniform labeling of intact tissues by synchronizing the labeling reaction globally. We first screened a range of chemicals for controlling the binding kinetics of the lipophilic dye and discovered that 10 mM SDS effectively inhibits staining (FIG. 5B). This result indicates that buffers containing 10 mM

SDS might have a potential to be used as a “SWITCH-Off” buffer. Using an approach analogous to SWITCH-mediated GA fixation, we thought it might be possible to allow dye molecules to disperse uniformly throughout a sample in the SWITCH-Off buffer and then activate global probe-target binding with the SWITCH-On buffer (FIG. 5D).

To test this approach, we first incubated a 1-mm-thick mouse brain block in PBST containing 10 mM SDS and lipophilic dyes for 24 hr at 37° C. (SWITCH-Off step). Then, we moved the tissue to PBST and incubated it for 3 hr at 37° C. (SWITCH-On step). The result was strikingly uniform labeling of all the myelinated axons within the sample (FIG. 5D). Myelinated fibers were clearly visible throughout the depth while the control tissue showed signal only from the surface (FIG. 5C).

We leveraged this fiber visualization capability to investigate how fibers and fascicles are organized in a mouse brain. Previous research has shown that fibers may be organized in 3D grids (Wedeen et al., 2012). However, the structure of all of the individual fibers has not yet been studied at the microscopic resolutions and macroscopic scales necessary to visualize their 3D organization. To that end, we obtained a volume image of labeled myelinated fibers in a SWITCH-processed mouse brain coronal slice spanning from the cortex to the striatum (FIG. 5E). This volume shows three main orientations of the fibers organized in a cubic grid: one radially projecting from the corpus callosum and two parallel to the corpus callosum. These three orientations are all orthogonal to one another (FIG. 5F). The volume also shows fascicles that radiate from the striatum and diverge, almost at right angles, at the corpus callosum (FIG. 5E). To quantify this finding in a non-biased manner, we determined the orientation of each of the fibers present in the volume and calculated the angles at which these fibers would intersect (FIG. 5G). In all three dimensions, the fibers indeed oriented themselves approximately orthogonally to each other (FIG. 5H). We used a similar approach to examine the fascicle orientations and found that they diverge almost orthogonally with respect to the corpus callosum in one of the axes (FIG. 5I). These results are corroborated by the autocorrelation results. This finding was made possible by the high-resolution and large-volume visualization capability of our method. A low-resolution approach would overlook the individual fibers while a low-volume approach would be unable to capture the entire connectional anatomy.

We then tested whether this application of SWITCH could be scaled to larger tissues. We applied the SWITCH approach for labeling an intact mouse hemisphere, but with 4 days of incubation in PEST containing 10 mM SDS and lipophilic dyes (SWITCH-Off step) and 1 day in PBST (SWITCH-On step). We imaged this larger volume using a custom-built, high-speed light-sheet microscope (Tomer et al., 2012; Tomer et al., 2014) within 2 hours and observed uniform labeling of all myelinated fibers across the entire tissue (FIG. 5J). As demonstrated, the SWITCH-labeling approach is scalable to organ-scale tissues. Just by scaling the incubation time with respect to the tissue size, we were able to label the whole tissue. The cost of the dye molecules used for labeling the hemisphere was less than one dollar. We also demonstrated that this approach can be used for visualizing myelinated fibers in spinal cords. These results show that the SWITCH-labeling method can be used to uniformly label tissues ranging from a 1-mm-thick block to an entire hemisphere for quantitative analysis.

### SWITCH Enables Scalable and Uniform Antibody Labeling

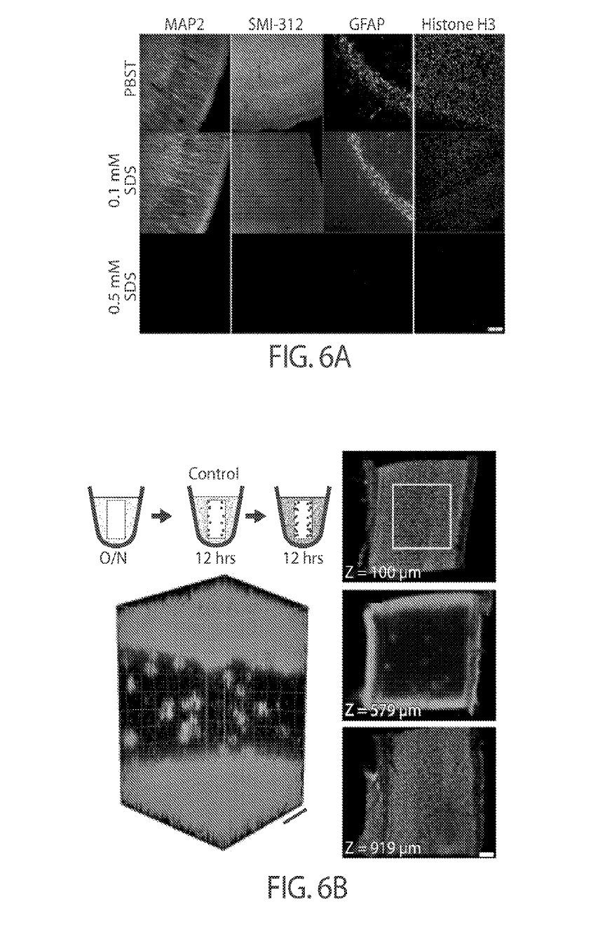

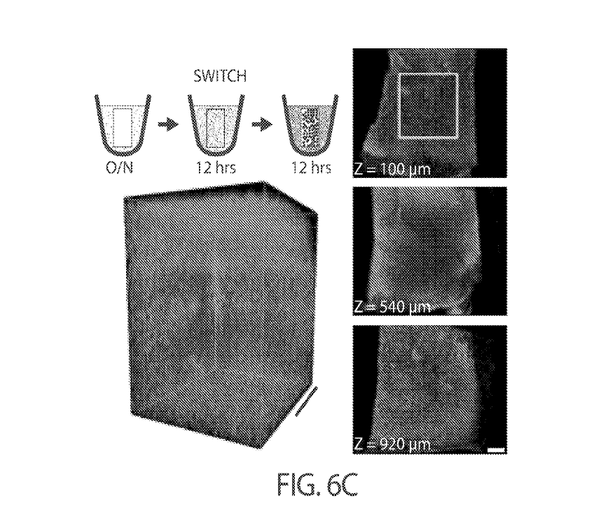

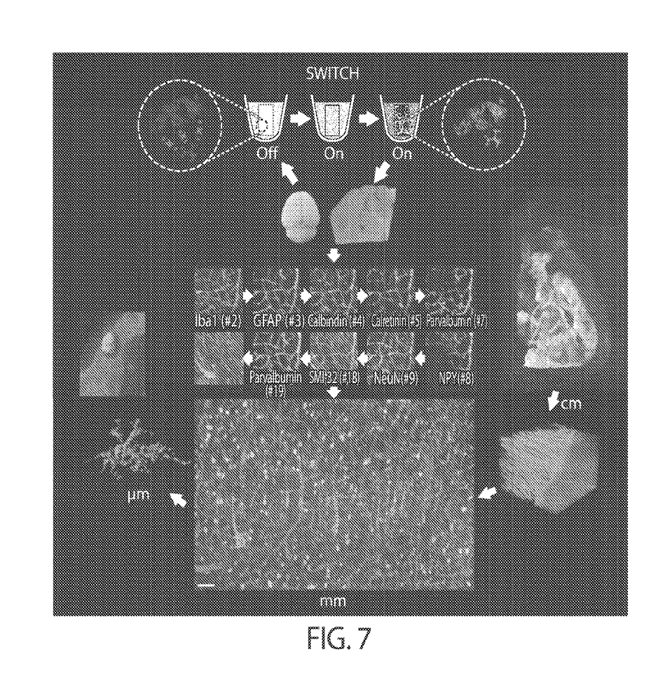

We then asked whether SWITCH-mediated labeling could be applied to the use of antibodies. We hypothesized that SDS could again be used as an effective inhibitor of antibody-antigen binding in small concentrations. Indeed, when we assayed for antibody labeling at various concentrations of SDS, we found that 0.5 to 1.0 mM was a high enough concentration to inhibit binding for many antibodies (FIG. 6A).

Based on the results of our binding assay, we chose PBS containing 0.5 mM SDS as a SWITCH-Off buffer and PBST as a SWITCH-On buffer. We hypothesized that, because very little antibody-antigen binding is occurring in the SWITCH-Off condition, antibodies would effectively be able to diffuse to equilibrium throughout the sample more rapidly than in PBST, in which antibodies are rapidly depleted at the surface (FIG. 6B). To test this, we attempted to label 1-mm-thick mouse brain blocks using anti-histone H3 antibodies. We labeled one sample using a 12-hr SWITCH-Off/12-hr SWITCH-On cycle and another using a standard immunohistochemistry protocol with 12 hr of primary antibody incubation in PBST followed by a 12 hr wash. For the SWITCH-On step, antibodies were not added to PBST. The result was a large increase in penetration depth and overall signal uniformity in the SWITCH sample relative to the control (FIG. 6B and FIG. 6C).

### Discussion

We have developed SWITCH, a simple method that enables scalable proteomic imaging of intact systems without requiring any specialized equipment or reagents. SWITCH is complementary to and thus may be used together with many pioneering technologies, each of which has its own unique advantages. For example, matrix-assisted laser desorption ionization mass spectrometry (MALDI-MS) and laser-ablation inductively coupled plasma mass spectrometry (LA-ICP-MS) allow visualization of a large subset of proteins and other biomolecules without a priori knowledge of targets. Recent advances in imaging mass spectrometry combined with immunohistochemistry (IHC) have significantly improved resolution (Angelo et al., 2014; Giesen et al., 2014), which was limited in MALDI-MS and LA-ICP-MS. This approach remarkably demonstrated analysis of more than 100 targets at subcellular resolution.

Multiplexing strategies for IHC that rely on iterative staining and elution have been developed. Among several pioneering techniques is array tomography, which involves cutting a tissue sample into tens or hundreds of nanometer-thick sections for staining and imaging (Micheva et al., 2010). These sections can be repeatedly washed and stained for probing different proteins. This powerful method yields subcellular resolution images of a small volume of tissue with fairly high multiplexing capability. Although these advanced technologies enable new approaches in studying complex biological systems, these methods require specialized equipment and are, therefore, difficult to implement in most labs.

With the aim of developing a simple and scalable method for proteomic imaging of both large animal and human samples, we first needed to devise the SWITCH method for controlling a broad range of chemical reactions in tissue processing to achieve uniform sample treatment regardless of tissue size and type. SWITCH dynamically modulates chemical reaction kinetics to synchronize the reaction time between molecules throughout the system. This strategy enables all endogenous molecular targets in a large intact tissue to experience similar reaction conditions (time and concentration). As a result, large tissues can be uniformly processed.

The SWITCH approach takes advantage of the way certain chemicals can be reversibly and rapidly changed by simply modulating their surrounding environment. For instance, in the GA-tissue-gelling step, we were able to decrease the rate of GA-biomolecule crosslinking by two orders of magnitude by using pH 3 buffer, because primary amine groups in endogenous biomolecules are protonated at low pH and the resulting charged amine cannot react with GA (Hopwood, 1972). This pH-dependent reactivity means that after uniformly dispersing GA in a tissue at low pH, we can “switch-on” inactivated amine groups by changing the amine's surrounding environment to a neutral-pH buffer. At neutral pH, charged amine groups are rapidly deprotonated and become reactive. In the case of human samples or animal samples that were previously PFA-fixed for a different purpose, this simple strategy enables all the endogenous biomolecules in a large intact tissue to simultaneously experience a similar GA-fixation/gelling condition. PFA-fixed tissues can withstand treatment at low pH while GA molecules are introduced. In the case of non-fixed samples, we recommend that they first be fixed with PFA before exposure to acidic conditions. In some instances, perfusion may be used.

Uniform GA-tissue-gel formation is an important first step towards our goal. Fixation of large samples via traditional immersion is unlikely to uniformly preserve them because highly reactive GA molecules are depleted within the outer layers of a sample. This presents a significant problem for iterative staining-based methods that rely on the removal of imaged probes using harsh elution conditions, because non-uniform preservation results in non-uniform loss of structure and molecules throughout the process. As demonstrated, our pH-SWITCH strategy ensures exceptionally uniform preservation of biological tissues that cannot be perfused (e.g., banked human clinical samples), meeting the requirements of proteomic imaging and quantitative phenotyping.

It has been noted that fixation with GA results in an increase in broad spectrum autofluorescence. While this autofluorescence has been low enough to allow quantitative analysis, it could be problematic in visualizing targets with low copy number. We investigated the use of sodium horohydride as a method of reducing autofluorescence, but found that the tissue damage resulting from this incubation procedure offset any benefits obtained from the modest decrease in autofluorescence that we were able to observe.

The use of reducing agents has allowed us to eliminate the issue of tissue browning during high-temperature clearing, but we also observed that excessive use of these chemicals may cause gradual tissue weakening. This is likely due to the reduction of disulfide linkages that maintain the tertiary structure of proteins within a sample, resulting in increased protein denaturation. Protein denaturation may lead to reduced sample antigenicity, but we have not found this to be an issue when using conservative amounts of reducing agents. Additionally, due to the instability of mRNA at elevated temperatures, this method of rapid clearing is not compatible with methods that require the preservation of mRNA.

Multiplexed imaging requires software to warp each experiment into a common coordinate system despite the subtle physical differences between each staining round. Variance can come in the form of rigid body changes (rotation, translation, and scale), illumination artifacts, stain quality, and tissue degradation. We observed that a feature-based algorithm gives maximum robustness across these sources of variance at the cost of increased computational requirements—a reasonable trade given the declining costs of such resources. To simplify the process, gross rigid alignments (i.e., rotating the tissue 180 degrees) are still best handled by human eye before the data is passed to the algorithm to achieve the cellular-scale registration.

SWITCH can provide a reliable way to obtain integrated high-dimensional information from intact biological samples. Using the cross-talk-free dataset, we successfully performed non-biased combinatorial expression analysis of a single human clinical tissue to unequivocally identify diverse cell-types based on their distinct protein expression patterns. Our quantitative analysis shows that CR+/PV+ cells do not exist within the examined volume of the human V2 cortex. The same finding was reported in mouse visual cortex (Gonchar et al., 2007), but such co-expression patterns among calcium-binding proteins may differ among brain regions and between individuals and species (Anelli and Heckman, 2006; DeFelipe et al.. 1999), which, therefore, calls for more comprehensive large-scale investigation.

We observed many NeuN-negative interneurons. NeuN, a neuron-specific RNA-binding protein known as Rbfox3 protein (Kim et al., 2009), has been widely used as a pan-neuronal marker for statistical analysis of many types of mature neurons (Baleriola et al., 2014; Pickrell et al., 2015). Only a few types of neurons are exceptions, such as cerebellar Purkinje cells, olfactory bulb mitral cells, and retinal photoreceptor cells (Mullen et al., 1992). However, even though we applied strict criteria to prevent weak NeuN+ cells from being identified as NeuN− cells, substantial portions of CB+, CR+, and PV+ neurons were still NeuN− while all SMI+ neurons were NeuN+. This result is supported by a recent report that some CR+ are not NeuN+, and CR and NeuN immunoreactivities have a negative correlation in the avian brainstem (Bloom et al., 2014) Likewise, in our experiments on human visual association cortex, cells with strong immunoreactivity against calcium-binding protein markers were frequently negative or very weakly positive for NeuN. These findings, together with a series of exceptional reports such as those on NeuN+cultured astrocytes (Darlington et al., 2008) and GFAP+ neuron-like cells (Oka et al., 2015), indicate that classical cell-type markers, particularly NeuN, may need to be used more carefully in light of their selectivity and function.

The SWITCH method has the potential to modulate a wide range of probe-target binding reactions. Probe-target interactions are governed by a multiplicity of non-covalent bonds such as hydrogen bonds, electrostatic forces, van der Waals bonds, and hydrophobic interactions (Mian et al., 1991). These weak forces can be effectively controlled by changing the surrounding chemical environment (e.g., ionic strength, pH, chemical additive, and temperature) (Kamata et al., 1996). For instance, we discovered that the addition of SDS alone, in different concentrations, can completely inhibit lipophilic dye-target and antibody-antigen binding reactions.

The SWITCH method's unique uniform-labeling capability enables quantitative analysis of large tissues that was previously only possible for thin tissue sections. Quantitative analysis relies heavily on signal intensity and SNR. Non-uniform or heterogeneous labeling would prohibit or, even worse, bias the analysis. While post hoc image processing methods could correct for small gradients in labeling (or imaging), large gradients caused by non-uniform labeling, where the surface of the tissue is saturated while the core is mostly unlabeled, would preclude image recovery. If the labeling is heterogeneous, the resulting data would be heavily biased, and no image processing methods could salvage such data in a fair way. This is why quantitative analysis of non-uniformly labeled tissues is a great challenge. However, tissues labeled using SWITCH exhibit uniform signal intensity and SNR throughout the tissue. Such a clear dataset lends itself well to quantitative analysis.

Although SWITCH enables processing of large samples, the speed of labeling is still fundamentally limited by passive diffusion. This is not of concern for smaller samples or even single-round investigation of large samples, but multiplexed imaging of large samples becomes impractical as a result, potentially taking months or years to collect the range of desired data. Recently developed methods of stochastic electrotransport (Kim et al., 2015) could potentially be combined with SWITCH to facilitate these experiments.

Together with its simplicity, scalability, and broad applicability, our data suggest that SWITCH provides access to high-dimensional multi-scale information that may help to understand health and disease from molecules to cells to entire systems.

### REFERENCES

1. Alivisatos, A. P., Chun, M., Church, G. M., Deisseroth, K., Donoghue, J. P., Greenspan, R. J., McEuen, P. L., Roukes, M. L., Sejnowski, T. J., Weiss, P. S., et al. (2013). The brain activity map. Science 339,1284-1285.
2. Anelli, R., and Heckman, C. J. (2006). The calcium binding proteins calbindin, parvalbumin, and calretinin have specific patterns of expression in the gray matter of cat spinal cord. J. Neurocytol. 34,369-385.
3. Angelo, M., Bendall, S. C., Finck, R., Hale, M. B., Hitzman, C., Borowsky, A. D.,

Levenson, R. M., Lowe, J. B., Liu, S. D., Zhao, S., et al. (2014). Multiplexed ion beam imaging of human breast tumors. Nat. Med. 20,436-442.
4. Baleriola, J., Walker, C.A., Jean, Y. Y., Crary, J. F., Troy, C. M., Nagy, P. L., and Hengst, U. (2014). Axonally Synthesized ATF4 Transmits a Neurodegenerative Signal across Brain Regions. Cell 158,1159-1172.
5. Bookstein, F. L. (1989). Principal warps: thin-plate splines and the decomposition of deformations. IEEE Trans. Pattern Anal. Mach. Intell. 11, 567-585.
6. Bloom, S., Williams, A., and MacLeod, K. M. (2014). Heterogeneous calretinin expression in the avian cochlear nucleus angularis. J. Assoc. Res. Otolaryngol. 15,603-620.
7. Campbell, M. J., and Morrison, J. H. (1989). Monoclonal antibody to neurofilament protein (SMI-32) labels a subpopulation of pyramidal neurons in the human and monkey neocortex. J. Comp. Neurol. 282,191-205.
8. Chung, K., and Deisseroth, K. (2013). CLARITY for mapping the nervous system. Nat. Methods 10,508-513.
9. Chung, K., Wallace, J., Kim, S.-Y., Kalyanasundaram, S., Andalman, A. S., Davidson, T. J., Mirzabekov, J. J., Zalocusky, K. a, Mattis, J., Denisin, A. K., et al. (2013). Structural and molecular interrogation of intact biological systems. Nature 497,332-337.
10. Darlington, P. J., Goldman, J. S., Cui, Q. L., Antel, J. P., and Kennedy, T. E. (2008). Widespread immunoreactivity for neuronal nuclei in cultured human and rodent astrocytes. J. Neurochem. 104,1201-1209.
11. DeFelipe, J., Gonzalez-Albo, M. C., Del Rio. M. R., and Elston, G. N. (1999). Distribution and patterns of connectivity of interneurons containing calbindin, calretinin, and parvalbumin in visual areas of the occipital and temporal lobes of the macaque monkey. J. Comp. Neurol. 412,515-526.
12. Fischler, M. A., and Bolles, R. C. (1981). Random sample consensus: a paradigm for model fitting with applications to image analysis and automated cartography. Commun. ACM 24, 381-395.
13. Friedman, M. (1996). Food Browning and Its Prevention : An Overview. J. Agric. Food Chem. 44,631-653.
14. Giesen, C., Wang, H. A. O., Schapiro, D., Zivanovic, N., Jacobs, A., Hattendorf, B., Schüffler, P. J., Grolimund, D., Buhmann, J. M., Brandt, S., et al. (2014). Highly multiplexed imaging of tumor tissues with subcellular resolution by mass cytometry. Nat. Methods 11, 417-422.
15. Gonchar, Y., Wang, Q., and Burkhalter, A. (2007). Multiple distinct subtypes of GABAergic neurons in mouse visual cortex identified by triple immunostaining. Front. Neuroanat. 1,3.
16. Harris. C., and Stephens, M. (1988). A Combined Corner and Edge Detector. Proc. Alvey Vis. Conf. 1988 147-151.
17. Hopwood, D. (1967). Some aspects of fixation with glutaraldehyde. A biochemical and histochemical comparison of the effects of formaldehyde and glutaraldehyde fixation on various enzymes and glycogen, with a note on penetration of glutaraldehyde into liver. J. Anat. 101,83-92.
18. Hopwood, D. (1970). The reactions between formaldehyde, glutaraldehyde and osmium tetroxide, and their fixation effects on bovine serum albumin and on tissue blocks. Histochemie 24,50-64.
19. Hopwood, D. (1972). Theoretical and practical aspects of glutaraldehyde fixation. Histochem. J. 4,267-303.
20. Kamata, N., Enomoto, A., Ishida, S., Nakamura, K., Kurisaki, J.-I., and Kaminogawa, S. (1996). Comparison of pH and ionic strength dependence of interactions between monoclonal antibodies and bovine β-lactoglobulin. Biosci. Biotech. Biochem. 60, 25-29.
21. Kasthuri, N., Hayworth, K. J., Berger, D. R., Schalek, R. L., Conchello, J. A., Knowles-Barley, S., Lee, D., Vazquez-Reina, A., Kaynig, V., Jones, T.R., et al. (2015). Saturated Reconstruction of a Volume of Neocortex. Cell 162,648-661.
22. Keller, S., Heerklotz, H., and Blume, A. (2006). Monitoring lipid membrane translocation of sodium dodecyl sulfate by isothermal titration calorimetry. J. Am. Chem. Soc. 128, 1279-1286.
23. Kim. K. K., Adelstein, R. S., and Kawamoto, S. (2009). Identification of neuronal nuclei (NeuN) as Fox-3, a new member of the Fox-1 gene family of splicing factors. J. Biol. Chem. 284, 31052-31061.
24. Kim, S.-Y., Cho, J. H., Murray, E., Bakh, N., Choi, H., Ohn, K., Ruelas, L., Hubbert, A., McCue, M., Vassallo, S.L., et al. (2015). Stochastic electrotransport selectively enhances the transports of highly electromobile molecules. PNAS. DOI:10.1073/pnas.1510133112.
25. Leuba, G., Kraftsik, R., and Saini, K. (1998). Quantitative distribution of parvalbumin, calretinin, and calbindin D-28k immunoreactive neurons in the visual cortex of normal and Alzheimer cases. Exp. Neurol. 152, 278-291.
26. Lowe, D. G. (2004). Distinctive Image Features from Scale-Invariant Keypoints. Int. J. Comp. Vis. 60, 91-110.
27. Manzo, C., van Zanten, T. S., Saha, S., Torreno-Pina, J. A., Mayor, S., and Garcia-Parajo, M. F. (2014). PSF decomposition of nanoscopy images via Bayesian analysis unravels distinct molecular organization of the cell membrane. Sci. Rep. 4, 4354.
28. McCaslin, A. F. H., Chen, B. R., Radosevich, A. J., Cauli, B., and Hillman, E. M. C. (2011). In vivo 3D morphology of astrocyte-vasculature interactions in the somatosensory cortex: implications for neurovascular coupling. J. Cereb. Blood Flow Metab. 31, 795-806.
29. Mian, I. S., Bradwell, A. R., and Olson, A. J. (1991). Structure, function and properties of antibody binding sites. J. Mol. Biol. 217, 133-151.
30. Micheva, K. D., Busse, B., Weiler, N. C., O'Rourke, N., and Smith, S. J. (2010). Single-synapse analysis of a diverse synapse population: Proteomic imaging methods and markers. Neuron 68, 639-653.
31. Mullen, R. J., Buck, C. R., and Smith, A. M. (1992). NeuN, a neuronal specific nuclear protein in vertebrates. Development 116, 201-211.
32. Oka, Y., Ye, M., and Zuker, C. S. (2015). Thirst driving and suppressing signals encoded by distinct neural populations in the brain. Nature 520, 349-352.
33. Pickrell, A. M., Huang, C.-H., Kennedy, S. R., Ordureau, A., Sideris, D. P., Hoekstra, J. G., Harper, J. W., and Youle, R. J. (2015). Endogenous Parkin Preserves Dopaminergic Substantia Nigral Neurons following Mitochondrial DNA Mutagenic Stress. Neuron 87, 371-381.
34. Rah, J.-C., Bas, E., Colonell, J., Mishchenko, Y., Karsh, B., Fetter, R. D., Myers, E. W., Chklovskii, D. B., Svoboda, K., Harris, T. D., et al. (2013). Thalamocortical input onto layer 5 pyramidal neurons measured using quantitative large-scale array tomography. Front. Neural Circuits 7, 177.
35. Renier, N., Wu, Z., Simon. D. J., Yang, J., Ariel, P., and Tessier-lavigne, M. (2014). Resource iDISCO : A Simple , Rapid Method to Immunolabel Large Tissue Samples for Volume Imaging. Cell 159, 896-910.
36. Richardson, D. S., and Lichtman, J. W. (2015). Clarifying Tissue Clearing. Cell 162, 246-257.
37. Roozemond, R. C. (1969). The effect of fixation with formaldehyde and glutaraldehyde on the composition of phospholipids extractable from rat hypothalamus. J. Histochem. Cytochem. 17,482-486.
38. Schindelin, J., Arganda-Carreras, I., Frise, E., Kaynig, V., Longair, M., Pietzsch, T., Preibisch, S., Rueden, C., Saalfeld, S., Schmid, B., et al. (2012). Fiji: an open-source platform for biological-image analysis. Nat. Methods 9, 676-682.
39. Schlessinger, J., Axelrod, D., Koppel, D. E., Webb, W. W., and Elson, E. L. (1977). Lateral transport of a lipid probe and labeled proteins on a cell membrane. Science 195, 307-309.
40. Scovanner, P., Ali, S., and Shah, M. (2007). A 3-dimensional sift descriptor and its application to action recognition. 15th Int. Conf. Multimed. 357-360.
41. De Sousa, A. A., Sherwood, C. C., Schleicher, A., Amunts, K., MacLeod, C. E., Hof, P. R., and Zilles, K. (2010). Comparative cytoarchitectural analyses of striate and extrastriate areas in hominoids. Cereb. Cortex 20,966-981.
42. Steinman, L. (1999). Assessment of animal models for MS and demyelinating disease in the design of rational therapy. Neuron 24,511-514.
43. Sung, H., Hsu, H., Shih, C., and Lin, D. (1996). Cross-linking characteristics of biological tissues fixed with monofunctional or multifunctional epoxy compounds. Biomaterials 17,1405-1410.
44. Susaki, E. A., Tainaka, K., Perrin, D., Kishino, F., Tawara, T., Watanabe, T. M., Yokoyama, C.. Onoe, H., Eguchi, M., Yamaguchi, S., et al. (2014). Whole-brain imaging with single-cell resolution using chemical cocktails and computational analysis. Cell 157,726-739.
45. Thomas, C., Ye, F. Q., Irfanoglu, M. O., Modi, P., Saleem, K. S., Leopold, D. A., and Pierpaoli, C. (2014). Anatomical accuracy of brain connections derived from diffusion MRI tractography is inherently limited. Proc. Natl. Acad. Sci. U.S.A. 111,16574-16579.
46. Tomer, R., Khairy, K., Amat. F., and Keller, P. J. (2012). Quantitative high-speed imaging of entire developing embryos with simultaneous multiview light-sheet microscopy. Nat. Methods 9,755-763.
47. Tomer, R., Ye, L., Hsueh, B., and Deisseroth, K. (2014). Advanced CLARITY for rapid and high-resolution imaging of intact tissues. Nat. Protocols 9,1682-1697.
48. Wedeen, V. J., Rosene, D. L., Wang, R., Dai, G., Mortazavi, F., Hagmann, P., Kaas, J.H., and Tseng, W.-Y.I. (2012). The geometric structure of the brain fiber pathways. Science 335,1628-1634.
49. Yuste, R. (2015). From the neuron doctrine to neural networks. Nat. Rev. Neurosci. 16, 1-11.
50. Zuccaro, E., and Arlotta, P. (2013). The quest for myelin in the adult brain. Nat. Cell Biol. 15,572-575.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also he understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall he closed or semi-closed transitional phrases, respectively, as set forth in the United States Patent Office Manual of Patent Examining Procedures, Section 2111.03.

What is claimed is:

1. A method for preserving a biological sample comprising
contacting a biological sample with a crosslinker under a first condition that reduces crosslinking activity at least 100-fold, and
then exposing the sample to a second condition that restores crosslinking activity,
wherein the first condition minimally comprises a low pH, and the second condition minimally comprises a neutral pH.

2. A method for processing a sample comprising
exposing a sample to a first condition comprising a crosslinker and low pH, and
exposing the sample to a second condition comprising neutral pH, and
then inactivating the crosslinker.

3. A method for preserving a sample comprising
exposing a sample to a crosslinker under a first condition that reduces crosslinking activity at least 100-fold, and
exposing the sample to a second condition that restores cros slinking activity, and
then inactivating the crosslinker.

4. The method of claim 3, wherein the first condition comprises low pH.

5. The method of claim 3, wherein the second condition comprises neutral pH.

6. The method of any one of the foregoing claims, wherein low pH is a pH of about 3.

7. The method of any one of the foregoing claims, wherein neutral pH is a pH of about 7.

8. The method of any one of the foregoing claims, wherein the first condition comprises a temperature of about 4-10° C.

9. The method of any one of the foregoing claims, wherein the first condition comprises a temperature of about 4° C.

10. The method of any one of the foregoing claims, wherein the second condition comprises a temperature of about 25-40° C.

11. The method of any one of the foregoing claims, wherein the second condition comprises a temperature of about 37° C.

12. The method of any one of the foregoing claims, wherein the sample is exposed to the crosslinker under the first condition for 1-2 days.

13. The method of any one of the foregoing claims, wherein the sample is exposed to the second condition for 1-10 hours.

14. The method of any one of the foregoing claims, wherein the crosslinker is a bifunctional crosslinker.

15. The method of any one of the foregoing claims, wherein the crosslinker is a multifunctional crosslinker.

16. The method of any one of the foregoing claims, wherein the crosslinker is ethylene glycol diglycidyl ether (EGDGE), dipropylene glycol diglycidyl ether (GE23), 1,4-butanediol diglycidyl ether (GE21), glycerol polyglycidyl ether (EX-313), or glutaraldehyde (GA).

17. The method of any one of the foregoing claims, wherein the sample is a human tissue sample.

18. The method of any one of the foregoing claims, wherein the sample is an animal tissue sample.

19. The method of any one of the foregoing claims, wherein the sample is a brain, liver, lung, kidney or spinal cord sample.

20. The method of any one of the foregoing claims, wherein the sample is contacted with a solution of about 4-10% crosslinker under the first condition.

21. The method of any one of the foregoing claims, wherein the sample is contacted with a solution of about 1-4% crosslinker under the second condition.

22. The method of any one of the foregoing claims, wherein the crosslinker is inactivated by addition of glycine and acetamide.

23. A method for imaging a sample comprising
(1) preserving a sample according to the method of any one of claims 1-22,
(2) contacting the sample with one or more binding partners, each binding partner specific for a cellular or extracellular target,
(3) detecting binding partners bound to the sample by obtaining an image of the sample,
(4) clearing the binding partners from the sample, and
(5) repeating steps (2) through (4) one or more times.

24. The method of claim 23, wherein steps (2) through (4) are repeated at least 10 times, or at least 20 limes.

25. The method of claim 23 or 24, wherein the sample is not substantially degraded throughout the method.

26. The method of any one of claims 23-25, wherein the images obtained are overlayed and aligned to obtain a composite image.

27. The method of any one of claims 23-26, wherein the sample is cleared using high temperature.

28. The method of any one of claims 23-26, wherein the sample is cleared using a temperature of about 80° C., optionally for 1-4 days.

29. The method of any one of claims 23-28, wherein the binding partners are antibodies or antigen-binding antibody fragments.

30. The method of any one of claims 23-29, wherein the targets are myelinated axons and fibers.

31. The method of any one of claims 23-30, further comprising detecting nucleic acids and/or lectins in the sample.

32. The method of any one of claims 23-31, wherein the sample is contacted with binding partners for DNA, lectin and a target in step (2).

33. The method of any one of claims 23-32, wherein the sample is incubated with reducing agents.

34. The method of claim 33, wherein the reducing agents are sodium sulfite and 1-thioglycerol.

35. A method for preserving a biological sample comprising
contacting a biological sample with a crosslinker under a first condition comprising a pH in the range of about 6 to about 8, and
then exposing the biological sample to a second condition comprising a higher pH.

36. A method for processing a sample comprising
exposing a sample to a first condition comprising a crosslinker and a pH in the range of about 6 to about 8, and
then exposing the sample to a second condition comprising a higher pH, and
then inactivating the crosslinker.

37. The method of claim 35 or 36, wherein the second condition comprises a pH of about 8 to about 11, or about 9 to about 11, or about 10 to about 11, or about 9 to about 10.

38. The method of any one of claims 35-37, wherein the first condition comprises a temperature of about 4-10° C., or about 4° C.

39. The method of any one of claims 35-38, wherein the second condition comprises a temperature of about 25-40° C., or about 37° C.

40. The method of any one of claims 35-39, wherein the sample is exposed to the crosslinker under the first condition for 1-2 days and/ or the sample is exposed to the second condition for 1-10 hours.

41. The method of any one of claims 35-40, wherein the crosslinker is a bifunctional crosslinker or a multifunctional crosslinker, optionally wherein the crosslinker is ethylene glycol diglycidyl ether (EGDGE), dipropylene glycol diglycidyl ether (GE23), 1,4-butanediol diglycidyl ether (GE21), glycerol polyglycidyl ether (EX-313), polyglycerol-3-polyglycidyl ether (GE38), or glutaraldehyde (GA).

42. The method of any one of claims 35-41, wherein the sample is a human tissue sample or an animal tissue sample, optionally wherein the sample is a brain, liver, lung, kidney or spinal cord sample.

43. The method of any one of claims 35-42, wherein the sample is contacted with a solution of about 4-10% crosslinker under the first condition and/or the sample is contacted with a solution of about 1-4% crosslinker under the second condition.

44. The method of any one of claims 35-43, wherein the crosslinker is inactivated by addition of glycine and acetamide.

45. A method for imaging a sample comprising
(1) preserving a sample according to the method of any one of claims 35-44,
(2) contacting the sample with one or more binding partners, each binding partner specific for a cellular or extracellular target,
(3) detecting binding partners bound to the sample by obtaining an image of the sample,
(4) clearing the binding partners from the sample, and
(5) repeating steps (2) through (4) one or more times.

46. The method of claim 45, wherein steps (2) through (4) are repeated at least 10 times, or at least 20 times.

47. The method of claim 45 or 46, wherein the sample is not substantially degraded throughout the method.

48. The method of any one of claims 45-47, wherein the images obtained are overlayed and aligned to obtain a composite image.

49. The method of any one of claims 45-48, wherein the sample is cleared using high temperature, optionally wherein the sample is cleared using a temperature of about 80° C., optionally for 1-4 days.

50. The method of any one of claims 45-49, wherein the binding partners are antibodies or antigen-binding antibody fragments.

51. The method of any one of claims 45-50, wherein the targets are myelinated axons and fibers.

52. The method of any one of claims 45-51, further comprising detecting nucleic acids and/or lectins in the sample, optionally wherein the sample is contacted with binding partners for DNA and lectin and with a binding partner for target in step (2).

53. The method of any one of claims 45-52, wherein the sample is incubated with reducing agents, optionally wherein the reducing agents are sodium sulfite and 1-thioglycerol.
